SEQUENCE LISTING (5 sequences)

SEQ ID NO: 1 - DNA, 4729 bp, Sugarcane
  o-methyltransferase 2 (SHOMT2) promoter (1)..(4726)
  Promoter (1)..(4726): o-methyltransferase 2 (SHOMT2) promoter
gctgtcgacgcggccgcgtaatacgactcactatagggcgaagaattcggatcttcatgtgtctcagaattgctctttatatgaactacttttgcatcaactgtccatctatttagtacgtattgttcaggaatttttttgatattgtttatatcaagtatcttcaacgcatggcagcataagattccaacaaattcaaactttttgcagctacacttcacctcaacttcggatgcagaaaactttacaacatgtttcttaccataaacctttattttgtactccttttcagcattactgtcaccacaacagaacagatcacaattcagtgtttgcatcacttgttcttggaataccttaaatatggctggagtataaattcttgttgcatgccttagtattctcaagtcagatttcaacttaggtgtactttgtgttgccttgaaatcacattttacctcctcatatcttttatctgccaccaacctttcaaagtgctcaaagaaggtaagtatgtcatatttgacactaataataccccttcaattcattgttcatgctttcacttctttgagtgctaaccatatcagcagaaaatgtatttcttccatatgctaaggcccatttctcccttttatcaaataatctttggagccattgattgtttcgtagcttatacttgtctagcaaattgttccatgcatttataaaatcctcctcctcctcttgatcataaatacatttttgaaaatctttattgaacttcttatagtcctccacaactccagcaaggtgcttgcacgcatttgtttcatatgccaaatacaaattctgtgatgagtctctggtagtgctatcttgattgcttttgccattgctgcatcttcatcggttaaaatggtttgtggcttcttgcctgacatagcagttaaaaatgtgtagaaaagccagacaaaactctcagcagtttcatcatacaaaagtgcagcaccaaaaattgttgttttcttgtgattattcactcccactagcaaaccaaatggacgtccgtcatttaattttctgtatgttgtgtcaaagcaaacaacatcaccaaaaacttcatagtctgcaaccattttagaatctgtccaaaaaatatttgttatcaagtcatcctcatcaacctgaatggaatagaaaaacttcccatcttctgacgttttcttctgtagatattccaatactcctcctgtgtccccatggtttatctgtagtgacctctttgaatacatgtttattcttcatatcttcacgagtaaatccaagattctcaactccacctgcttcttttgccatgaggatcaaaggtagctttatttgaaatgccgactgctttagcattttctacacttgcaagttgtgcttctgttattcttctttgagatcttaaatgatgaacttgactgctagtagcaagaatatgattatgttcaggctcaaactcatagatgcagtacaatccatctgtaagacttatcttcatgcgagcttcacacatacatcgtgtctctggtttactatagttggaagattcttcttttttatcaggacgccgaacacctattgataaataaataattgatcaaaatgatgttctatccctaatgatgaagaaagtggcacaagacaaaagtgatttaatacctgcacgagaacaacaaaaggttctattctttatggtggtgctatttttcacattatgtgaactgctccttcgaatactaaaacccttgtctcgagcataggcattgtaaaagcggtatgcctcttcttcagtgcagaatttcataccaaccttaggtatcctgtcttccatagaattttctacctgagtaggttcggtctggttggatttgtagcgggtttcatgcaaaataagttagaaatcgtgcaaacttgcaatggaggttaaatttcaaatatatttgcatagacaaaacaaatatagattatgaatggtaatccaatatgcatgacttgcattttctaactctattgctactgtgccagatgaagaatgttgatctggagaagttttgtgagaatgtgacaacaacgggaggtcatatcaagattctgggtacccgcggagaatcggcctccatgtagttagcctcgtcaggcatggggggaattggctgagatgcccccatgtagtcgtcaggcatggagagtactggctgagatgccattgttgtgtagatcgagagaaacgagaagaatgctagtctaataatacccttccgtatgtatctaaccaactattataattggcaccatttttcacatgctagcgccttttgcctgctttatttaattcaattgggtccgataagcatgtgaacgtgggagacggttccgtcggacggctccattttcttgtagcgtacggcgtggacggagaaaaggtgagggcccatctctgaaggggaacgaatggatggtggacacgtgtggggagacaccgaagggacatgccgaggaggcacacaagcttcagcaggcgtctccagactctcagaagaagaagaagctcacggcacggttgcggctggttcttgctgtcgctgtctcgtggtgcacgtttctgtgatcacgctgaaatcgaccggccggcggaccaacaggaggtcagctcggccactccgtctccgagcgcatgagtgcaccgttcgtccgcggttccttttctcgtggtgccgtgcacgcctctgcgttcaccggcaccctgaaaccaatcagaacgttccctttacaggggaaagggacaagtctgataacctctctgtttccatcgtcctctaaccgcgaagagcggacgcacaagacttagagtctatttgttcgaaattttttactctcacaaaagctagcttttatagacgggcataaaagctatcatgtcgaccggcacgtttaatatttaacttataccatatgaatatcatgtcaaactatgaggatgatacttttctgaacgtgattgcgtgagttattaaattgtacttttagttgtttgagcatgaaggtctgaactatgaatttatgatgtattgtggcttgtgagctactccgctctacatttagttggtatcataaatattattatattatcatataaatttgatcaaactttgactcttcaagattcttggaatgacttatcatttggggtagggagtaggtttctaaggccagtctcagtggggttttatcagagtttcatggacattaaataagctgatgtgacaccgtattgatgaagagagagatgataagagtttcatgagagtagagagagttttatggggatgaaactcttcttcactgtttccaaaatatagatgcattggtaagagggccatgaaatctctagtgacactgacctaagatgagattgactctagcactatgtttcaaaatctgcatgcatgcatgctttgaatattgtaacctcacattaactcccctcacacatgcatttgaatattgtaacctcacattaactcccctcacacatgcatgcaaacgggcggtgcacgcaaaagaattgagtgaagatgcacatgaaaaataagtaaaatgctttggcttcatcacccggcttaaatgctcgacagaaaaacacgtcggtagtcaaggttgtgcctaacaaactggggttcacatgtaaaacacgttcatgccttagaaacggcctggagggattagatacaacttcaattatatcttagggcccctccaatattgccagctctaaactagttttatgtgtcacggtggaggagagggaggctaaaaatataatcttgagctaacgtgaagagaagagctatttttttgctccccaatacatgagagatacaacatgagagaaaaatatatgaataaagaacactttacatgccagccatacaatatgagatttcatctaagagccaacacctgactcgtactgttgaaggtgtcctagttggagtggtcgatcttttagttgttagtagtgtaagacctagtttagtgctcttttcttgtctaggtttatgttgtgttttggctgccaagtgttgaacaactcaaggtaaggtcccatctaattctaaaatgatgccaaataaagatagattacaaagttaaacgacgggaaaactctaaaataggatggaaagttttgtagagtaataattggtacgaagtggcgaagtcgaccacaaccaaacataaagagttaaatgcatggtaggctcttgatcttgtctggaggtgccacttaggtccacaaactctcaaattgcatttttgacaccctaatgttattcaagtgtgccacttagatccacaaactctcaaaatgcatttctggtaccctagtgttgttcaagtgtgtcacttaggcaagaaaagttagataattttgataagctatgggaccaaattaatttatgtatgcatgctcgaactagttgatgatgatggaccccataatagacactagttcgtgggctggtttccttgtatagtactagctagtataactttttcaagttgtagctactactttagcttatactccgcatattacaatcaaatagaattcggaagtactataaacgggagcctataaatggagacgttttgcatcatgaggctataacaacttgagcaaaaacagaagccgtgcgccatg

SEQ ID NO: 2 - DNA, 6868 bp, Artificial Sequence
  Expression cassette SHOMT2-GUS-NOS
gctgtcgacgcggccgcgtaatacgactcactatagggcgaagaattcggatcttcatgtgtctcagaattgctctttatatgaactacttttgcatcaactgtccatctatttagtacgtattgttcaggaatttttttgatattgtttatatcaagtatcttcaacgcatggcagcataagattccaacaaattcaaactttttgcagctacacttcacctcaacttcggatgcagaaaactttacaacatgtttcttaccataaacctttattttgtactccttttcagcattactgtcaccacaacagaacagatcacaattcagtgtttgcatcacttgttcttggaataccttaaatatggctggagtataaattcttgttgcatgccttagtattctcaagtcagatttcaacttaggtgtactttgtgttgccttgaaatcacattttacctcctcatatcttttatctgccaccaacctttcaaagtgctcaaagaaggtaagtatgtcatatttgacactaataataccccttcaattcattgttcatgctttcacttctttgagtgctaaccatatcagcagaaaatgtatttcttccatatgctaaggcccatttctcccttttatcaaataatctttggagccattgattgtttcgtagcttatacttgtctagcaaattgttccatgcatttataaaatcctcctcctcctcttgatcataaatacatttttgaaaatctttattgaacttcttatagtcctccacaactccagcaaggtgcttgcacgcatttgtttcatatgccaaatacaaattctgtgatgagtctctggtagtgctatcttgattgcttttgccattgctgcatcttcatcggttaaaatggtttgtggcttcttgcctgacatagcagttaaaaatgtgtagaaaagccagacaaaactctcagcagtttcatcatacaaaagtgcagcaccaaaaattgttgttttcttgtgattattcactcccactagcaaaccaaatggacgtccgtcatttaattttctgtatgttgtgtcaaagcaaacaacatcaccaaaaacttcatagtctgcaaccattttagaatctgtccaaaaaatatttgttatcaagtcatcctcatcaacctgaatggaatagaaaaacttcccatcttctgacgttttcttctgtagatattccaatactcctcctgtgtccccatggtttatctgtagtgacctctttgaatacatgtttattcttcatatcttcacgagtaaatccaagattctcaactccacctgcttcttttgccatgaggatcaaaggtagctttatttgaaatgccgactgctttagcattttctacacttgcaagttgtgcttctgttattcttctttgagatcttaaatgatgaacttgactgctagtagcaagaatatgattatgttcaggctcaaactcatagatgcagtacaatccatctgtaagacttatcttcatgcgagcttcacacatacatcgtgtctctggtttactatagttggaagattcttcttttttatcaggacgccgaacacctattgataaataaataattgatcaaaatgatgttctatccctaatgatgaagaaagtggcacaagacaaaagtgatttaatacctgcacgagaacaacaaaaggttctattctttatggtggtgctatttttcacattatgtgaactgctccttcgaatactaaaacccttgtctcgagcataggcattgtaaaagcggtatgcctcttcttcagtgcagaatttcataccaaccttaggtatcctgtcttccatagaattttctacctgagtaggttcggtctggttggatttgtagcgggtttcatgcaaaataagttagaaatcgtgcaaacttgcaatggaggttaaatttcaaatatatttgcatagacaaaacaaatatagattatgaatggtaatccaatatgcatgacttgcattttctaactctattgctactgtgccagatgaagaatgttgatctggagaagttttgtgagaatgtgacaacaacgggaggtcatatcaagattctgggtacccgcggagaatcggcctccatgtagttagcctcgtcaggcatggggggaattggctgagatgcccccatgtagtcgtcaggcatggagagtactggctgagatgccattgttgtgtagatcgagagaaacgagaagaatgctagtctaataatacccttccgtatgtatctaaccaactattataattggcaccatttttcacatgctagcgccttttgcctgctttatttaattcaattgggtccgataagcatgtgaacgtgggagacggttccgtcggacggctccattttcttgtagcgtacggcgtggacggagaaaaggtgagggcccatctctgaaggggaacgaatggatggtggacacgtgtggggagacaccgaagggacatgccgaggaggcacacaagcttcagcaggcgtctccagactctcagaagaagaagaagctcacggcacggttgcggctggttcttgctgtcgctgtctcgtggtgcacgtttctgtgatcacgctgaaatcgaccggccggcggaccaacaggaggtcagctcggccactccgtctccgagcgcatgagtgcaccgttcgtccgcggttccttttctcgtggtgccgtgcacgcctctgcgttcaccggcaccctgaaaccaatcagaacgttccctttacaggggaaagggacaagtctgataacctctctgtttccatcgtcctctaaccgcgaagagcggacgcacaagacttagagtctatttgttcgaaattttttactctcacaaaagctagcttttatagacgggcataaaagctatcatgtcgaccggcacgtttaatatttaacttataccatatgaatatcatgtcaaactatgaggatgatacttttctgaacgtgattgcgtgagttattaaattgtacttttagttgtttgagcatgaaggtctgaactatgaatttatgatgtattgtggcttgtgagctactccgctctacatttagttggtatcataaatattattatattatcatataaatttgatcaaactttgactcttcaagattcttggaatgacttatcatttggggtagggagtaggtttctaaggccagtctcagtggggttttatcagagtttcatggacattaaataagctgatgtgacaccgtattgatgaagagagagatgataagagtttcatgagagtagagagagttttatggggatgaaactcttcttcactgtttccaaaatatagatgcattggtaagagggccatgaaatctctagtgacactgacctaagatgagattgactctagcactatgtttcaaaatctgcatgcatgcatgctttgaatattgtaacctcacattaactcccctcacacatgcatttgaatattgtaacctcacattaactcccctcacacatgcatgcaaacgggcggtgcacgcaaaagaattgagtgaagatgcacatgaaaaataagtaaaatgctttggcttcatcacccggcttaaatgctcgacagaaaaacacgtcggtagtcaaggttgtgcctaacaaactggggttcacatgtaaaacacgttcatgccttagaaacggcctggagggattagatacaacttcaattatatcttagggcccctccaatattgccagctctaaactagttttatgtgtcacggtggaggagagggaggctaaaaatataatcttgagctaacgtgaagagaagagctatttttttgctccccaatacatgagagatacaacatgagagaaaaatatatgaataaagaacactttacatgccagccatacaatatgagatttcatctaagagccaacacctgactcgtactgttgaaggtgtcctagttggagtggtcgatcttttagttgttagtagtgtaagacctagtttagtgctcttttcttgtctaggtttatgttgtgttttggctgccaagtgttgaacaactcaaggtaaggtcccatctaattctaaaatgatgccaaataaagatagattacaaagttaaacgacgggaaaactctaaaataggatggaaagttttgtagagtaataattggtacgaagtggcgaagtcgaccacaaccaaacataaagagttaaatgcatggtaggctcttgatcttgtctggaggtgccacttaggtccacaaactctcaaattgcatttttgacaccctaatgttattcaagtgtgccacttagatccacaaactctcaaaatgcatttctggtaccctagtgttgttcaagtgtgtcacttaggcaagaaaagttagataattttgataagctatgggaccaaattaatttatgtatgcatgctcgaactagttgatgatgatggaccccataatagacactagttcgtgggctggtttccttgtatagtactagctagtataactttttcaagttgtagctactactttagcttatactccgcatattacaatcaaatagaattcggaagtactataaacgggagcctataaatggagacgttttgcatcatgaggctataacaacttgagcaaaaacagaagccgtgcgccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccgggatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgcc

SEQ ID NO: 3 - DNA, 7063 bp, Artificial Sequence
  Expression cassette SHOMT2-GUS first exon/catalase intron/GUS second exon, His6-NOS
gctgtcgacgcggccgcgtaatacgactcactatagggcgaagaattcggatcttcatgtgtctcagaattgctctttatatgaactacttttgcatcaactgtccatctatttagtacgtattgttcaggaatttttttgatattgtttatatcaagtatcttcaacgcatggcagcataagattccaacaaattcaaactttttgcagctacacttcacctcaacttcggatgcagaaaactttacaacatgtttcttaccataaacctttattttgtactccttttcagcattactgtcaccacaacagaacagatcacaattcagtgtttgcatcacttgttcttggaataccttaaatatggctggagtataaattcttgttgcatgccttagtattctcaagtcagatttcaacttaggtgtactttgtgttgccttgaaatcacattttacctcctcatatcttttatctgccaccaacctttcaaagtgctcaaagaaggtaagtatgtcatatttgacactaataataccccttcaattcattgttcatgctttcacttctttgagtgctaaccatatcagcagaaaatgtatttcttccatatgctaaggcccatttctcccttttatcaaataatctttggagccattgattgtttcgtagcttatacttgtctagcaaattgttccatgcatttataaaatcctcctcctcctcttgatcataaatacatttttgaaaatctttattgaacttcttatagtcctccacaactccagcaaggtgcttgcacgcatttgtttcatatgccaaatacaaattctgtgatgagtctctggtagtgctatcttgattgcttttgccattgctgcatcttcatcggttaaaatggtttgtggcttcttgcctgacatagcagttaaaaatgtgtagaaaagccagacaaaactctcagcagtttcatcatacaaaagtgcagcaccaaaaattgttgttttcttgtgattattcactcccactagcaaaccaaatggacgtccgtcatttaattttctgtatgttgtgtcaaagcaaacaacatcaccaaaaacttcatagtctgcaaccattttagaatctgtccaaaaaatatttgttatcaagtcatcctcatcaacctgaatggaatagaaaaacttcccatcttctgacgttttcttctgtagatattccaatactcctcctgtgtccccatggtttatctgtagtgacctctttgaatacatgtttattcttcatatcttcacgagtaaatccaagattctcaactccacctgcttcttttgccatgaggatcaaaggtagctttatttgaaatgccgactgctttagcattttctacacttgcaagttgtgcttctgttattcttctttgagatcttaaatgatgaacttgactgctagtagcaagaatatgattatgttcaggctcaaactcatagatgcagtacaatccatctgtaagacttatcttcatgcgagcttcacacatacatcgtgtctctggtttactatagttggaagattcttcttttttatcaggacgccgaacacctattgataaataaataattgatcaaaatgatgttctatccctaatgatgaagaaagtggcacaagacaaaagtgatttaatacctgcacgagaacaacaaaaggttctattctttatggtggtgctatttttcacattatgtgaactgctccttcgaatactaaaacccttgtctcgagcataggcattgtaaaagcggtatgcctcttcttcagtgcagaatttcataccaaccttaggtatcctgtcttccatagaattttctacctgagtaggttcggtctggttggatttgtagcgggtttcatgcaaaataagttagaaatcgtgcaaacttgcaatggaggttaaatttcaaatatatttgcatagacaaaacaaatatagattatgaatggtaatccaatatgcatgacttgcattttctaactctattgctactgtgccagatgaagaatgttgatctggagaagttttgtgagaatgtgacaacaacgggaggtcatatcaagattctgggtacccgcggagaatcggcctccatgtagttagcctcgtcaggcatggggggaattggctgagatgcccccatgtagtcgtcaggcatggagagtactggctgagatgccattgttgtgtagatcgagagaaacgagaagaatgctagtctaataatacccttccgtatgtatctaaccaactattataattggcaccatttttcacatgctagcgccttttgcctgctttatttaattcaattgggtccgataagcatgtgaacgtgggagacggttccgtcggacggctccattttcttgtagcgtacggcgtggacggagaaaaggtgagggcccatctctgaaggggaacgaatggatggtggacacgtgtggggagacaccgaagggacatgccgaggaggcacacaagcttcagcaggcgtctccagactctcagaagaagaagaagctcacggcacggttgcggctggttcttgctgtcgctgtctcgtggtgcacgtttctgtgatcacgctgaaatcgaccggccggcggaccaacaggaggtcagctcggccactccgtctccgagcgcatgagtgcaccgttcgtccgcggttccttttctcgtggtgccgtgcacgcctctgcgttcaccggcaccctgaaaccaatcagaacgttccctttacaggggaaagggacaagtctgataacctctctgtttccatcgtcctctaaccgcgaagagcggacgcacaagacttagagtctatttgttcgaaattttttactctcacaaaagctagcttttatagacgggcataaaagctatcatgtcgaccggcacgtttaatatttaacttataccatatgaatatcatgtcaaactatgaggatgatacttttctgaacgtgattgcgtgagttattaaattgtacttttagttgtttgagcatgaaggtctgaactatgaatttatgatgtattgtggcttgtgagctactccgctctacatttagttggtatcataaatattattatattatcatataaatttgatcaaactttgactcttcaagattcttggaatgacttatcatttggggtagggagtaggtttctaaggccagtctcagtggggttttatcagagtttcatggacattaaataagctgatgtgacaccgtattgatgaagagagagatgataagagtttcatgagagtagagagagttttatggggatgaaactcttcttcactgtttccaaaatatagatgcattggtaagagggccatgaaatctctagtgacactgacctaagatgagattgactctagcactatgtttcaaaatctgcatgcatgcatgctttgaatattgtaacctcacattaactcccctcacacatgcatttgaatattgtaacctcacattaactcccctcacacatgcatgcaaacgggcggtgcacgcaaaagaattgagtgaagatgcacatgaaaaataagtaaaatgctttggcttcatcacccggcttaaatgctcgacagaaaaacacgtcggtagtcaaggttgtgcctaacaaactggggttcacatgtaaaacacgttcatgccttagaaacggcctggagggattagatacaacttcaattatatcttagggcccctccaatattgccagctctaaactagttttatgtgtcacggtggaggagagggaggctaaaaatataatcttgagctaacgtgaagagaagagctatttttttgctccccaatacatgagagatacaacatgagagaaaaatatatgaataaagaacactttacatgccagccatacaatatgagatttcatctaagagccaacacctgactcgtactgttgaaggtgtcctagttggagtggtcgatcttttagttgttagtagtgtaagacctagtttagtgctcttttcttgtctaggtttatgttgtgttttggctgccaagtgttgaacaactcaaggtaaggtcccatctaattctaaaatgatgccaaataaagatagattacaaagttaaacgacgggaaaactctaaaataggatggaaagttttgtagagtaataattggtacgaagtggcgaagtcgaccacaaccaaacataaagagttaaatgcatggtaggctcttgatcttgtctggaggtgccacttaggtccacaaactctcaaattgcatttttgacaccctaatgttattcaagtgtgccacttagatccacaaactctcaaaatgcatttctggtaccctagtgttgttcaagtgtgtcacttaggcaagaaaagttagataattttgataagctatgggaccaaattaatttatgtatgcatgctcgaactagttgatgatgatggaccccataatagacactagttcgtgggctggtttccttgtatagtactagctagtataactttttcaagttgtagctactactttagcttatactccgcatattacaatcaaatagaattcggaagtactataaacgggagcctataaatggagacgttttgcatcatgaggctataacaacttgagcaaaaacagaagccgtgcgccatggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgactcgtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactcgaagtcacagccaaaagccagacagagtctgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttcagctgtctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaagctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 4 - DNA, 2910 bp, Sugarcane
  Promoter (1)..(2907): o-methyltransferase 1 (SHOMT1) promoter
gcataggcattgtaaaagcggtatgcctcttcttcagtgcagaatttcataccaaccttaggtatcctgtcttccatagaattttctacctgagtaggttcggtctggttggatttgtagcgggtttcatgcaaaataagttagaaatcgtgcaaacttgcaatggaggttaaatttgaaatatatttgcatagacaaaacaaatatagattatgaatggtaatccaatatgacttgcattttctaactctattgctactgtgccagatgaagaatgttgatctggagaagttttgtgagaatgtgacaacaacgggaggtcatatcaagattctgggtacccgcggagaatcggcctccatgtagttagcctcgtcaggcatggggggaattggctgagatgcccccatgtagtcgtcaggcatggagagtactggctgagatgccattgttgtgtagatcgagagaaacgagaagaatgctagtctaataatacccttccgtatgctaaccaactattataattggcaccatttttcacatgctagcgccttttgcctgctttatttaattcaattgggtccgataagcatgtgaacgtgggagacggttccgtcggacggctccgttttcttgtagcgtacggcgtggacggagaaaaggtgagggcctatctctaaaggggaacgaatggatggtggacacgtgtggggagacaccgaagggacatgccgaggaggcacacaagcttcagcaggcgtctccagactctcagaagaagaagaagctcacggcacggttgcggctggttcttgctgtcgctgtctcgtggtgcacgtttctgtgatcacgctgaaatcgaccggccggcggaccaacaggaggtcagctcggccactccgtctccgagcgcatgagtgcaccgttcgtccgcggttccttttctcgtggtgccgtgcacgcctctgcgttcaccggcaccctgaaaccaatcagaacgttccctttacaggggaaagggacaagtctgataacctctctgtttccatcgtcctctaaccgcgaagagcggacgcacaagacttagagtctatttgttcgaaattttttactctcacaaaagctagcttttatagacgggcataaaagctatcatgtcgaccggcacgtttaatatttaacttataccatatgaatatcatgtcgaactatgaggatgatacttttctgaacgtgattgcgtgagttattaaattgtacttttagttgtttgagcatgaaggtctgaactatgaatttatgatgtattgtggcttgtgagctactccgctctacatttagttggtatcataaatattattatattatcatataaatttgatcaacttgagatgctttgactcttcaagattcttggaatgacttatcatttggggtagggagtaggtttctaaggccagtctcagtggggtttcatcagagtttcatggacattaaataagctgatgtgacaccgtattgatgaagagagagatgataagagtttcatgcgagtagagagagtttcatggggatgaaactcttcttcactgtttccaaaatatagatgcattggtaagagggccatgaaatctctagtgacactgacctaagatgagattgactctagcactatgtttcaaaatctgcatgcatgcatgctttgaatattgtaacctcacattaactcccctcacacatgcatgcaaacgggcggtgcacgcaaaagaattgagtgaagatgcacatgaaaaataagtaaaatgctttggcttcatcacccggcttaaatgctcgacagaaaaacacgtcggtagtcaaggttgtgcctaacaaactggggttcacatgtaaaacacgttcatgccttagaaacggcctggagggattagatacaacttcaattatatcttagggcccctccaatattgtcagctctaaactagttttatgtgtcacggtggaggagagggaggctaaaaatataatcttgagctaacgtgaagagaagagctatttttttttgctccccaatacatgatagatacaatatgagagaaaaaatatatgaataaagaacactttacatgccagccatacaatatgagatttcatctaagagccaacacctgactcgtactgttgaaggtgtcctagttggagtggtcgatcttttagttgttagtagtgtaagacctagtttagtgctcttttcttgtctaggtttatgttgtgttttggctgccaagtgttgaacaactcaaggtaaggtcccatctaattctaaaatgatgccaaataaagatagattacaaagttaaacgacggaaaaactctaaaataggatggaaagttttgtagagtaataattggtatgaagtggcgaagtcgaccacaaccaaacataaagagttaaatgcatggtaggctcttgatcttgtctggaggtgccacttaggtccacaaactctcaaattgcatttttgacaccctaatgttattcaagtgtgccacttagatctacaaactctcaaaatgcatttctgataccctagtgttgttcaagtgtgtcacttaggcaagaaaagttagataattttgataagctatgggaccaaattaatttatgtatgcatgctcgaactagttgatgatgatggaccccataatagacactagttcatgggctggtttccttgtatagtactagctagtataactttttcaagttgtagctactactttagcttatactccgcatattacaatcaaatagaattcggaagtactataaacgggagcctataaatggagacgttttgcatcatgaggctataacaacttgagcaaaaacagaagccgtgcgccatg

SEQ ID NO: 5 - DNA, 2956 bp, Artificial Sequence
  An example embodiment of a SHOMT1 / SHOMT2 consensus sequence
gcataggcattgtaaaagcggtatgcctcttcttcagtgcagaatttcataccaaccttaggtatcctgtcttccatagaattttctacctgagtaggttcggtctggttggatttgtagcgggtttcatgcaaaataagttagaaatcgtgcaaacttgcaatggaggttaaatttsaaatatatttgcatagacaaaacaaatatagattatgaatggtaatccaatatnnnngacttgcattttctaactctattgctactgtgccagatgaagaatgttgatctggagaagttttgtgagaatgtgacaacaacgggaggtcatatcaagattctgggtacccgcggagaatcggcctccatgtagttagcctcgtcaggcatggggggaattggctgagatgcccccatgtagtcgtcaggcatggagagtactggctgagatgccattgttgtgtagatcgagagaaacgagaagaatgctagtctaataatacccttccgtatgnnnctaaccaactattataattggcaccatttttcacatgctagcgccttttgcctgctttatttaattcaattgggtccgataagcatgtgaacgtgggagacggttccgtcggacggctccrttttcttgtagcgtacggcgtggacggagaaaaggtgagggccyatctctraaggggaacgaatggatggtggacacgtgtggggagacaccgaagggacatgccgaggaggcacacaagcttcagcaggcgtctccagactctcagaagaagaagaagctcacggcacggttgcggctggttcttgctgtcgctgtctcgtggtgcacgtttctgtgatcacgctgaaatcgaccggccggcggaccaacaggaggtcagctcggccactccgtctccgagcgcatgagtgcaccgttcgtccgcggttccttttctcgtggtgccgtgcacgcctctgcgttcaccggcaccctgaaaccaatcagaacgttccctttacaggggaaagggacaagtctgataacctctctgtttccatcgtcctctaaccgcgaagagcggacgcacaagacttagagtctatttgttcgaaattttttactctcacaaaagctagcttttatagacgggcataaaagctatcatgtcgaccggcacgtttaatatttaacttataccatatgaatatcatgtcraactatgaggatgatacttttctgaacgtgattgcgtgagttattaaattgtacttttagttgtttgagcatgaaggtctgaactatgaatttatgatgtattgtggcttgtgagctactccgctctacatttagttggtatcataaatattattatattatcatataaatttgatcaannnnnnnngctttgactcttcaagattcttggaatgacttatcatttggggtagggagtaggtttctaaggccagtctcagtggggtttyatcagagtttcatggacattaaataagctgatgtgacaccgtattgatgaagagagagatgataagagtttcatgmgagtagagagagtttyatggggatgaaactcttcttcactgtttccaaaatatagatgcattggtaagagggccatgaaatctctagtgacactgacctaagatgagattgactctagcactatgtttcaaaatctgcatgcatgcatgctttgaannnnnnnnnnnnnnnnnnnnnnnnnnnnnnnnnnnnnnnnnntattgtaacctcacattaactcccctcacacatgcatgcaaacgggcggtgcacgcaaaagaattgagtgaagatgcacatgaaaaataagtaaaatgctttggcttcatcacccggcttaaatgctcgacagaaaaacacgtcggtagtcaaggttgtgcctaacaaactggggttcacatgtaaaacacgttcatgccttagaaacggcctggagggattagatacaacttcaattatatcttagggcccctccaatattgycagctctaaactagttttatgtgtcacggtggaggagagggaggctaaaaatataatcttgagctaacgtgaagagaagagctatttttttnngctccccaatacatgakagatacaatatgagagaaaaantatatgaataaagaacactttacatgccagccatacaatatgagatttcatctaagagccaacacctgactcgtactgttgaaggtgtcctagttggagtggtcgatcttttagttgttagtagtgtaagacctagtttagtgctcttttcttgtctaggtttatgttgtgttttggctgccaagtgttgaacaactcaaggtaaggtcccatctaattctaaaatgatgccaaataaagatagattacaaagttaaacgacggraaaactctaaaataggatggaaagttttgtagagtaataattggtaygaagtggcgaagtcgaccacaaccaaacataaagagttaaatgcatggtaggctcttgatcttgtctggaggtgccacttaggtccacaaactctcaaattgcatttttgacaccctaatgttattcaagtgtgccacttagatcyacaaactctcaaaatgcatttctgrtaccctagtgttgttcaagtgtgtcacttaggcaagaaaagttagataattttgataagctatgggaccaaattaatttatgtatgcatgctcgaactagttgatgatgatggaccccataatagacactagttcrtgggctggtttccttgtatagtactagctagtataactttttcaagttgtagctactactttagcttatactccgcatattacaatcaaatagaattcggaagtactataaacgggagcctataaatggagacgttttgcatcatgaggctataacaacttgagcaaaaacagaagccgtgcgcc

(19) United States
(12) Patent Application Publication
(10) Pub. No.: US 2013/0247252 A1
(43) Pub. Date: Sep. 19, 2013

(54) COMPOSITIONS, ORGANISMS, SYSTEMS, AND METHODS FOR EXPRESSING A GENE PRODUCT IN PLANTS

(71) Applicant: THE TEXAS A&M UNIVERSITY SYSTEM, College Station, TX (US)

(72) Inventors: Mona B. DAMAJ, Westlaco, TX (US); T. Erik MIRKOV, Harlingen, TX (US); Terry L. THOMAS, College Station, TX (US); Keerti S. RATHORE, College Station, TX (US); Chandrakanth EMANI, Owensboro, KY (US); Siva P. KUMPATLA, Carmel, IN (US)

(73) Assignee: THE TEXAS A&M UNIVERSITY SYSTEM, College Station, TX (US)

(21) Appl. No.: 13/800,930

(22) Filed: Mar. 13, 2013

Related U.S. Application Data
(60) Provisional application No. 61/612,744, filed on Mar. 19, 2012.

(51) Int. Cl. C12N 15/113

(52) U.S. Cl. CPC C12N 15/113
USPC 800/298; 536/24.1; 435/320.1; 435/252.3; 435/419; 435/412; 435/468; 435/469

(57) ABSTRACT

The present disclosure relates, according to some embodiments, to compositions, organisms, systems, and methods for expressing a gene product in a plant (e.g., a monocot) using a promoter operable in one or more plant tissues and/or cells. In some embodiments, an isolated nucleic acid may comprise an expression control sequence having the sequence of nucleotides 1-4726 of SEQ ID NO: 1, wherein the expression control sequence has stem-regulated and/or defense-inducible promoter activity in at least one monocot (e.g., at least two monocots).

### CROSS-REFERENCE TO RELATED APPLICATIONS

This application claims priority to U.S. Provisional Patent Application 61/612,744, filed on Mar. 19, 2012, which application is incorporated herein by reference.

### FIELD OF THE DISCLOSURE

The present disclosure relates, in some embodiments, to compositions, organisms, systems, and methods for expressing a gene product in a plant (e.g., a monocot) using a promoter operable in one or more plant tissues.

### BACKGROUND OF THE DISCLOSURE

Biotechnology promises to revolutionize everything from agriculture to modern medicine. For example, methods of genetically engineering plants are being explored to increase productivity through greater drought and insect resistance as well as increased yields. In addition, plants are being examined as potential biofactories for the production of proteins (e.g., antibodies) and other compounds for use in human and veterinary medicine. However, a limited number of expression control sequences (e.g., promoters) exist for driving expression of a gene product of interest in plants. Some of these are effective at driving expression in only some plants. Others are effective at driving expression in some tissues and/or cells, but not others.

### SUMMARY

Accordingly, a need has arisen for expression control sequences (e.g., promoters) operable in plants including promoters that are operable in monocots and/or promoters that are operable in one or more plant tissues and/or cells.

The present disclosure relates, according to some embodiments, to compositions, organisms, systems, and methods for expressing a gene product in a plant (e.g., a monocot) using a promoter operable in one or more plant tissues and/or cells. In some embodiments, an isolated nucleic acid may comprise an expression control sequence having the sequence of nucleotides 1-4726 of SEQ ID NO: 1, wherein the expression control sequence has stem-specific and/or defense-inducible promoter activity in at least one monocot (e.g., at least two monocots).

The present disclosure relates, in some embodiments, to an isolated nucleic acid comprising (a) an expression control sequence having the sequence of nucleotides 1-4726 of SEQ ID NO: 1, and (b) an exogenous nucleic acid (e.g., a transgene), wherein the expression control sequence has stem-specific and/or defense-inducible promoter activity in at least one monocot. An exogenous nucleic acid may alter carbon metabolism in the plant cell when expressed or transcribed in some embodiments. An exogenous nucleic acid may encode, in some embodiments, an insecticide effective against at least one stem-boring insect.

According to some embodiments, the present disclosure relates to an expression vector comprising, in a 5′ to 3′ direction: a sugarcane o-methyltransferase 2 (SHOMT2) promoter having a nucleotide sequence of nucleotides 1-4726 of SEQ ID NO: 1; an exogenous nucleic acid (e.g., a transgene); and a 3′ termination sequence, wherein the SHOMT2 promoter has stem-specific and/or defense-inducible promoter activity in at least one monocot. An expression vector may be located in a bacterial cell or a plant cell.

The present disclosure relates, in some embodiments, to a bacterial cell comprising an expression vector having: (a) a SHOMT2 promoter having a nucleotide sequence of nucleotides 1-4726 of SEQ ID NO: 1; (b) an exogenous nucleic acid; and (c) a 3′ termination sequence, wherein the SHOMT2 promoter has stem-specific and/or defense-inducible promoter activity in at least one monocot in some embodiments. The present disclosure further relates to a plant cell comprising an expression vector, in some embodiments, the expression vector comprising (a) a promoter having a nucleotide sequence of nucleotides 1-4726 of SEQ ID NO: 1; (b) an exogenous nucleic acid (e.g., a transgene) operably linked to the promoter; and (c) a 3′ termination sequence operably linked to the exogenous nucleic acid, wherein the promoter has stem-specific and/or defense-inducible promoter activity in at least one monocot. An exogenous nucleic acid may alter carbon metabolism in the plant cell when expressed or transcribed in some embodiments. An exogenous nucleic acid may encode, in some embodiments, an insecticide effective against at least one stem-boring insect. A plant cell comprising an expression vector may be located in a plant (e.g., a monocot) in some embodiments. Examples of a plant may include sugarcane, miscanthus, a miscanthus×sugarcane hybrid, switch grass, oat, wheat, barley, maize, rice, banana, yucca, onion, asparagus, sorghum and hybrids thereof.

According to some embodiments, the present disclosure relates to plants comprising an expression vector having: (a) a promoter having a nucleotide sequence of nucleotides 1-4726 of SEQ ID NO: 1; (b) an exogenous nucleic acid operably linked to the promoter; and (c) a 3′ termination sequence operably linked to the exogenous nucleic acid, wherein the promoter has stem-specific and/or defense-inducible promoter activity in at least one monocot. In addition, the present disclosure relates to methods for stem-specifically and/or defense-inducibly expressing an exogenous nucleic acid in a monocot, in some embodiments. For example, a method may comprise contacting an expression cassette or expression vector with the cytosol of a cell of the monocot, wherein the expression cassette or expression vector comprises (i) the exogenous nucleic acid, (ii) a SHOMT2 promoter comprising the sequence of nucleotides 1-4726 of SEQ ID NO: 1 and operable to drive expression of the exogenous nucleic acid in the monocot, and (iii) a 3′ termination sequence operably linked to the exogenous nucleic acid, and wherein the promoter has stem-specific and/or defense-inducible promoter activity in the monocot. In some embodiments, contacting further comprises biolistically bombarding the cell with a particle comprising the expression cassette or expression vector and/or co-cultivating the cell with an Agrobacterium cell comprising the expression cassette or expression vector. Plants in which an exogenous gene may be expressed include sugarcane, miscanthus, a miscanthus×sugarcane hybrid, switch grass, oat, wheat, barley, maize, rice, banana, yucca, onion, asparagus, sorghum and hybrids thereof.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

Some embodiments of the disclosure may be understood by referring, in part, to the present disclosure and the accompanying drawings, wherein:

FIG. 1 illustrates a sugarcane o-methyltransferase 2 promoter:13-glucuronidase expression vector (pSHOMT2GUSNOSpUC19) (SEQ ID NO:2) suitable for expression in sugarcane, maize and sorghum according to a specific example embodiment of the disclosure;

FIG. 2 illustrates a sugarcane o-methyltransferase 2 promoter:13-glucuronidase expression vector (pSHOMT2 pCAMBIA1301) (SEQ ID NO:3) suitable for expression in rice according to a specific example embodiment of the disclosure;

FIG. 3 illustrates a Southern blot analysis of XbaI digested DNA of seven sugarcane SHOMT positive genomic clones, using SHOMT full-length cDNA as a probe according to a specific example embodiment of the disclosure;

FIG. 4 illustrates a genomic Southern blot analysis of HindIII digested genomic DNA from two sugarcane lines transgenic for the β-glucuronidase (GUS) gene under the control of the sugarcane o-methyltransferase 2 (SHOMT2) promoter according to a specific example embodiment of the disclosure;

FIG. 5A illustrates a micrograph of transgenic sugarcane stems showing histochemical localization of the β-glucuronidase (GUS) gene expression driven by the sugarcane o-methyltransferase 2 (SHOMT2) promoter in the stem vasculature and storage parenchyma according to a specific embodiment of the disclosure;

FIG. 5B illustrates a micrograph of untransformed sugarcane stems showing no histochemical staining in the stem vasculature and storage parenchyma according to a specific embodiment of the disclosure;

FIG. 6 illustrates a Southern blot analysis of HindIII digested genomic DNA from two rice lines transgenic for the β-glucuronidase (GUS) gene under the control of a sugarcane o-methyltransferase 2 (SHOMT2) promoter according to a specific example embodiment of the disclosure;

FIG. 7A illustrates a micrograph of transgenic rice stems showing histochemical localization of the β-glucuronidase (GUS) gene expression driven by a sugarcane o-methyltransferase 2 promoter (SHOMT2) in the stem vasculature and storage parenchyma according to a specific embodiment of the disclosure;

FIG. 7B illustrates a micrograph of untransformed rice stems showing no histochemical staining in the stem vasculature and storage parenchyma according to a specific embodiment of the disclosure;

FIGS. 8A-8C illustrates a comparative micrograph of transgenic sugarcane stems according to specific embodiments of the disclosure in which,

FIG. 8A shows histochemical localization of the β-glucuronidase (GUS) gene expression driven by a sugarcane o-methyltransferase 2 (SHOMT2) promoter in the stem vasculature and storage parenchyma according to a specific embodiment of the disclosure,

FIG. 8B shows histochemical localization of the β-glucuronidase (GUS) gene expression driven by a sugarcane o-methyltransferase (SHOMT) promoter in the stem vasculature and storage parenchyma according to a specific embodiment of the disclosure, and

FIG. 8C shows histochemical localization of the β-glucuronidase (GUS) gene expression driven by a sugarcane dirigent 16 (SHDIR16) promoter in the stem vasculature and storage parenchyma according to a specific embodiment of the disclosure;

FIGS. 9A-9C illustrates a comparative micrograph of transgenic rice stems according to specific embodiments of the disclosure in which,

FIG. 9A shows histochemical localization of the β-glucuronidase (GUS) gene expression driven by a sugarcane o-methyltransferase 2 (SHOMT2) promoter in the stem vasculature and storage parenchyma according to a specific embodiment of the disclosure,

FIG. 9B shows histochemical localization of the β-glucuronidase (GUS) gene expression driven by a sugarcane o-methyltransferase (SHOMT) promoter in the stem vasculature according to a specific embodiment of the disclosure, and

FIG. 9C shows histochemical localization of the β-glucuronidase (GUS) gene expression driven by a sugarcane dirigent 16 (SHDIR16) promoter in the stem vasculature according to a specific embodiment of the disclosure; and

FIG. 10 illustrates an alignment of SHOMT1 (SEQ ID NO. 4) and SHOMT2 (SEQ ID NO. 1) according to a specific example embodiment of the disclosure.

### BRIEF DESCRIPTION OF THE SEQUENCE LISTING

Some embodiments of the disclosure may be understood by referring, in part, to the present disclosure and the accompanying sequence listing, wherein:

SEQ ID NO: 1 illustrates a sugarcane o-methyltransferase 2 promoter according to a specific example embodiment of the disclosure;

SEQ ID NO: 2 illustrates an expression cassette suitable for sugarcane transformation according to a specific example embodiment of the disclosure comprising a sugarcane o-methyltransferase 2 (SHOMT2) promoter, a β-glucuronidase (GUS) coding sequence, and an Agrobacterium nopaline synthase (NOS) terminator;

SEQ ID NO: 3 illustrates an expression cassette suitable for rice transformation according to a specific example embodiment of the disclosure comprising a sugarcane o-methyltransferase 2 (SHOMT2) promoter, a β-glucuronidase (GUS) coding sequence, and an Agrobacterium nopaline synthase (NOS) terminator;

SEQ ID NO: 4 illustrates a sugarcane o-methyltransferase 1 promoter according to a specific example embodiment of the disclosure; and

SEQ ID NO: 5 illustrates a sugarcane o-methyltransferase 1-o-methyltransferase 2 consensus sequence according to a specific example embodiment of the disclosure.

### DETAILED DESCRIPTION

The present disclosure relates, according to some embodiments, to compositions, organisms, systems, and methods for expressing a gene product in a plant (e.g., a monocot) using a promoter operable in one or more plant tissues and/or cells. For example, the present disclosure relates to expression control sequences (e.g., promoters), expression cassettes, expression vectors, microorganisms, and/or plants comprising a sugarcane o-methyltransferase 2 (SHOMT2) promoter. An expression control sequence, according to some embodiments, may be constitutively active or conditionally active in (a) an organ selected from root, leaf, stem, flower, seed, fruit, and/or tuber and/or (b) active in a tissue selected from epidermis, periderm, parenchyma, collenchyma, sclerenchyma, xylem, phloem, and/or secretory structures.

In some embodiments, an expression control sequence may be included in methods, compositions, systems, and/or organisms (a) to alter carbon metabolism (e.g., in a sucrose accumulating tissue) and/or (b) to express a protein (e.g., an insecticidal protein) in a plant (e.g., in sugarcane). An expression control sequence may be included, according to some embodiments, in methods, compositions, systems, and/or organisms to improve pest and/or disease tolerance and/or disease resistance (e.g., rice plants).

The disclosure, in some embodiments, relates to an expression control sequence operable in monocots (e.g., sugarcane, sorghum, maize, rice) to drive expression in one or more tissues (e.g., stem tissue). For example, an expression control sequence may comprise an isolated promoter of sugarcane that regulates expression of a gene for sugarcane o-methyltransferase (SHOMT2) protein. SHOMT2 protein may be involved in lignification and/or plant defense responses in some embodiments. A SHOMT2 expression control sequence may be stem-expressed according to some embodiments. A SHOMT2 expression control sequence may comprise a 4.726 kb nucleic acid region, which may be located upstream of the 5′ end of a sugarcane SHOMT2 structural coding sequence, and may be capable of driving high levels of gene and/or transgene expression in a stem-regulated manner in one or more plants (e.g., major agronomic crops such as sugarcane and rice).

According to some embodiments, a distinguishing feature of an expression control sequence over expression control sequences having a similar nucleic acid sequence may be operability in various organisms. For example, a first expression control sequence may be operable in as few as one species (e.g., the species from which it was originally isolated), whereas a second expression control sequences may be operable in two or more species. Operability may be assessed according to a variety of metrics including total transcript produced, total protein produced, cell and/or tissue types in which transcript is produced, cell and/or tissue types in which protein is produced, inducibility, among others. For example, some functional stem-expressed promoters may be available for use in transformation of sugarcane, an economically important crop, in terms of sucrose accumulation and biomass production. Such promoters may not be operable in a broader range of species, tissues, and/or cell types.

A finite number of expression control sequences are known to be operable in monocots (e.g., sugarcane, sorghum, maize, rice). Expression control sequences, according to the present disclosure, may supplement, complement, expand, and/or overcome perceived limits of the existing pool of monocot-operable expression control sequences. For example, expression control sequences, according to the present disclosure, may have one or more desirable features over other expression control sequences in regulating gene and/or transgene expression in the stem vasculature and/or storage parenchyma tissues.

Choice of an expression control sequence may influence (e.g., determine) when and/or where a gene of interest (operably linked to the expression control sequence) is expressed in a plant. The tissue-regulated expression conferred by a SHOMT2 promoter may be particularly important in maximizing metabolic energy into gene and/or transgene products at target sites, thereby reducing the impact on non-target tissues. A SHOMT2 promoter may be of value in engineering monocots for improved carbon metabolism for sugar accumulation and/or high fiber content for biofuel feedstock and bioenergy production, as well as for enhanced stress tolerance.

According to some embodiments, the present disclosure provides nucleic acid sequences and constructs, expression vectors, plant cells and transgenic plants comprising a SHOMT2 promoter. Transgenic plants (e.g., sugarcane, sorghum, maize, rice) may include a heterologous coding sequence operably linked to a SHOMT2. In some embodiments, expression of a heterologous coding sequence may be directed by the SHOMT2 promoter and may be limited to stem tissues. The disclosure relates, in some embodiments, to methods for producing nucleic acid vectors, expression cassettes and transgenic plants.

A SHOMT2 expression control sequence (e.g., promoter) may provide, in some embodiments, tight regulation of gene expression in stem tissues. According to some embodiments, a SHOMT2 expression control sequence may be inactive or substantially inactive in one or more (e.g., all) non-stem tissues of a plant. An expression control sequence (e.g., promoter) may drive expression of one or more genes/transgenes of interest at desirable levels and/or in desired target tissue(s). Regulated expression of genes and/or transgenes may ensure plant productivity, viability and/or fertility, for example, when constitutive expression of a gene/transgene is likely to compromise metabolism or important aspects of meristem or embryo function. Tissue-regulated expression may be desirable for increasing (e.g., maximizing) metabolic energy into gene/transgene products at target sites, thereby reducing the impact on non-target tissues. According to some embodiments, a SHOMT2 expression control sequence (e.g., promoter) may be less susceptible to silencing in one or more monocots than one or more existing stem-specific promoters. A SHOMT2 expression control sequence (e.g., promoter) may operate in one or more monocots including monocot crops (e.g., sugarcane, sorghum, maize, and rice).

According to some embodiments, the present disclosure relates to expression control sequences (e.g., regulatory sequences) operable to direct stem-regulated and/or defense-inducible expression. An expression control sequence may include promoters from a stem-expressed, defense-inducible family of genes (e.g., o-methyltransferase 2 (SHOMT2) genes). Expression control sequences, in some embodiments, may have specific advantages over other tissue-specific expression control sequences (e.g., promoters) in their enhanced specificity in regulating gene expression (a) in stem tissues and/or (b) in response to induction by external stimuli such as plant defense-inducing agents. Expression control sequences according to some embodiments of the disclosure may be very useful in methods for altering carbon metabolism in sucrose accumulating tissues and/or for driving expression of desired proteins (e.g., insecticidal proteins) in sugarcane. An expression control sequence (e.g., promoter) may also be included in methods of improved pest and/or disease tolerant plants (e.g., rice plants) in some embodiments.

The present disclosure relates to isolated nucleic acids, according to some embodiments, including promoters operable (e.g., primarily) in stem and/or in response to stimulation by defense-inducing agents. An expression control sequence (e.g., promoter) may hybridize (e.g., under stringent conditions) to an expression control sequence isolated from sugarcane (e.g., a SHOMT2 promoter).

### Expression Control Sequences

The disclosure relates, in some embodiments, to isolated nucleic acids including expression control sequences operable to direct stem-regulated and/or defense-inducible expression. The present disclosure relates, in some embodiments, to isolated nucleic acids comprising expression control sequences (e.g., promoters) capable of specifically directing expression in stem tissue and/or in response to stimulation by defense-inducing agents. For example, an expression control sequence (e.g., promoter), when operably linked to either a coding sequence of a gene or a sequence complementary to a native plant gene, may direct expression of the coding sequence or complementary sequence in stem tissue and/or in response to a defense-inducing agent.

In some embodiments, an SHOMT2 expression control sequence may be provided by screening a library of nucleic acids (e.g., a monocot genomic library) using an SHOMT2 nucleic acid, a fragment thereof, and/or a complement thereto as a probe. For example, an SHOMT2 promoter may be provided as follows. SHOMT2 recombinant genomic clones may be first isolated by screening a sugarcane genomic library constructed in bacteriophage Lambda DASH II vector with a cDNA (or a portion thereof) representing SHOMT2 mRNA. To obtain a cDNA representing SHOMT2 mRNA, a sugarcane stem-regulated cDNA library may be constructed and screened by differential hybridization with stem, leaf and root cDNA probes to identify stem-regulated cDNAs including the SHOMT2 cDNA. Sequences identical, similar, and/or homologous to SHOMT2 may be isolated using established cloning techniques and/or amplification techniques.

In some embodiments, an SHOMT2 expression control sequence (e.g., promoter) may be derived from restriction endonuclease digestion of isolated SHOMT2 genomic clones. For example, the nucleotide or amino acid sequence of the coding region of a gene of the o-methyl transferase gene family may be aligned to the nucleic acid or deduced amino acid sequence of an isolated stem-regulated genomic clone and the 5′ flanking sequence (i.e., sequence upstream from the translational start codon of the coding region) of the isolated SHOMT2 genomic clone may be located.

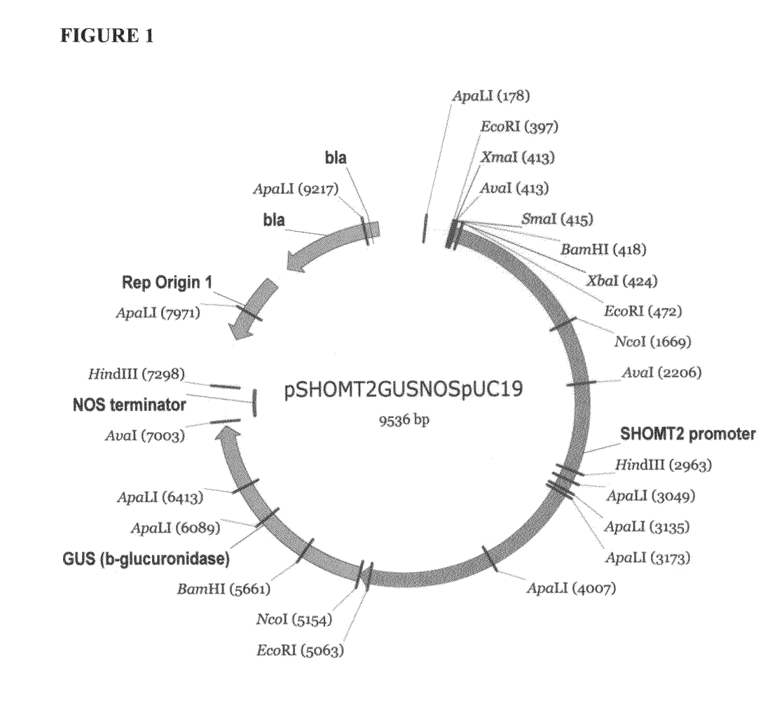

An SHOMT2 expression control sequence (e.g., promoter) as set forth in SEQ ID NO:1 (nucleotides −4726 to −1 of FIG. 1) may be generated, according to some embodiments, from genomic clones having either or both excess 5′ flanking sequence or coding sequence by exonuclease III-mediated deletion. This may be accomplished by digesting appropriately prepared DNA with exonuclease III (exoIII) and removing aliquots at increasing intervals of time during the digestion. The resulting successively smaller fragments of DNA may be sequenced to determine the exact endpoint of the deletions. Commercially available systems which use exonuclease III (exoIII) to create such a deletion series may include Promega Biotech, “Erase-A-Base”® system. Alternatively, PCR primers may be defined to allow direct amplification of an SHOMT2 expression control sequence (e.g., promoter).

In some embodiments, one or more deletion fragments of an SHOMT2 expression control sequence (e.g., SEQ ID NO:1) may be prepared using the same or similar methods. An expression control sequence may comprise at least one contiguous portion of the nucleotide sequences set forth in SEQ ID NO:1 and/or may be operable to direct stem-regulated and/or defense-inducible expression according to some embodiments.

An expression control sequence may include, in addition to a sugarcane SHOMT2 promoter having the nucleotide sequence of SEQ ID NO:1, sequences which correspond to the same gene, i.e., a homolog, in other plant species. Such related sequences which direct stem-regulated and/or defense-inducible expression, may be described in terms of their percent homology and/or identity on a nucleotide level to the nucleotide sequence of SEQ ID NO:1 in some embodiments. Such related sequences from other plant species may be defined in terms of their ability to hybridize to a nucleic acid having a nucleotide sequence of SEQ ID NO: 1 (or a fragment thereof larger than about 1 kb) under stringent hybridization conditions.

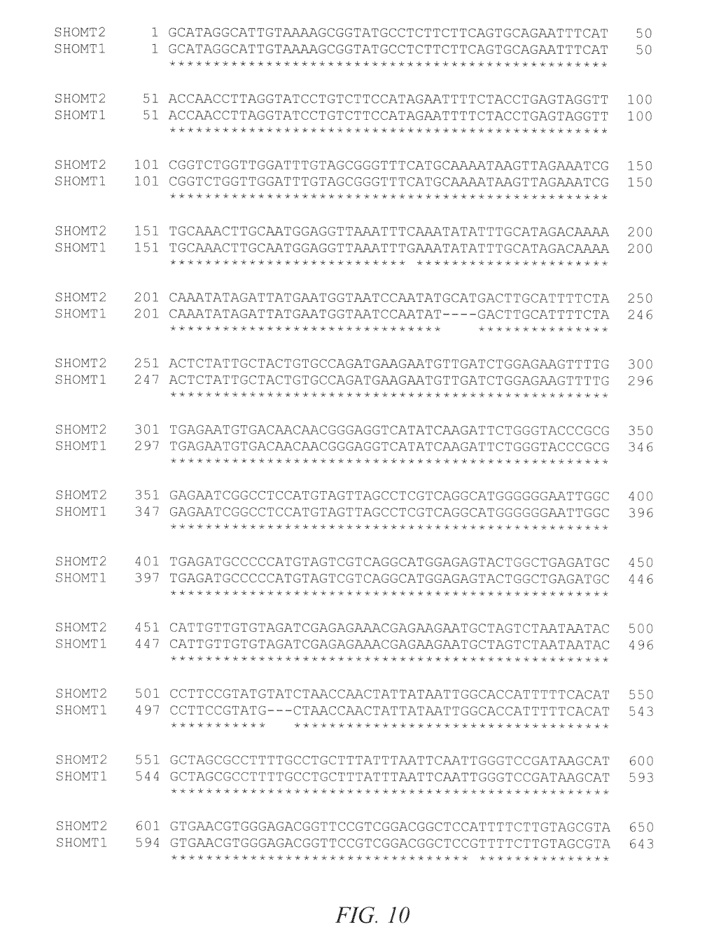

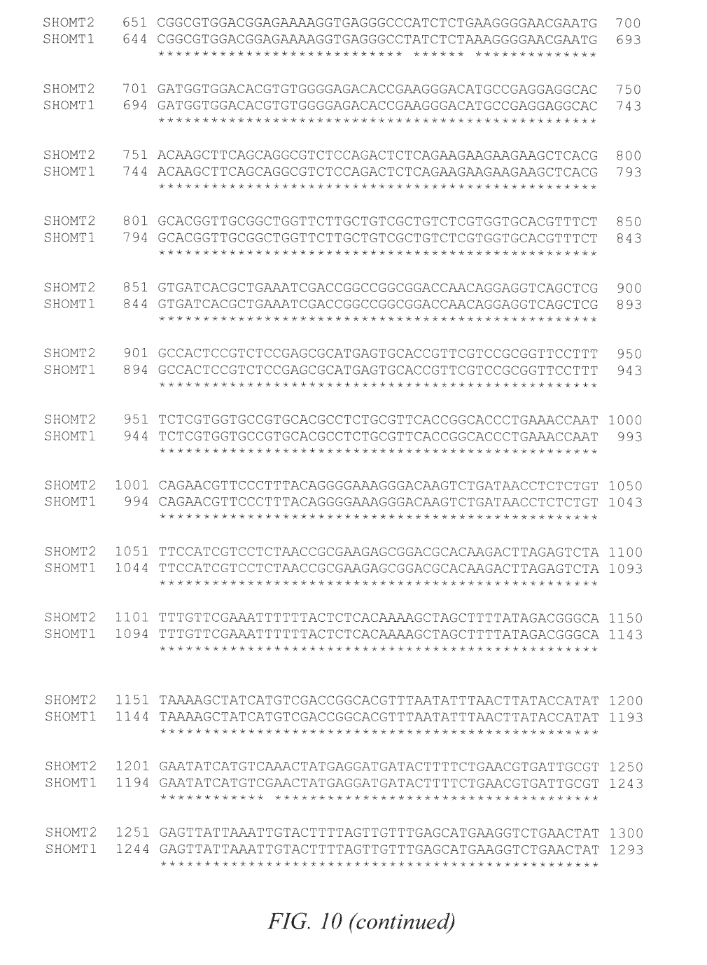

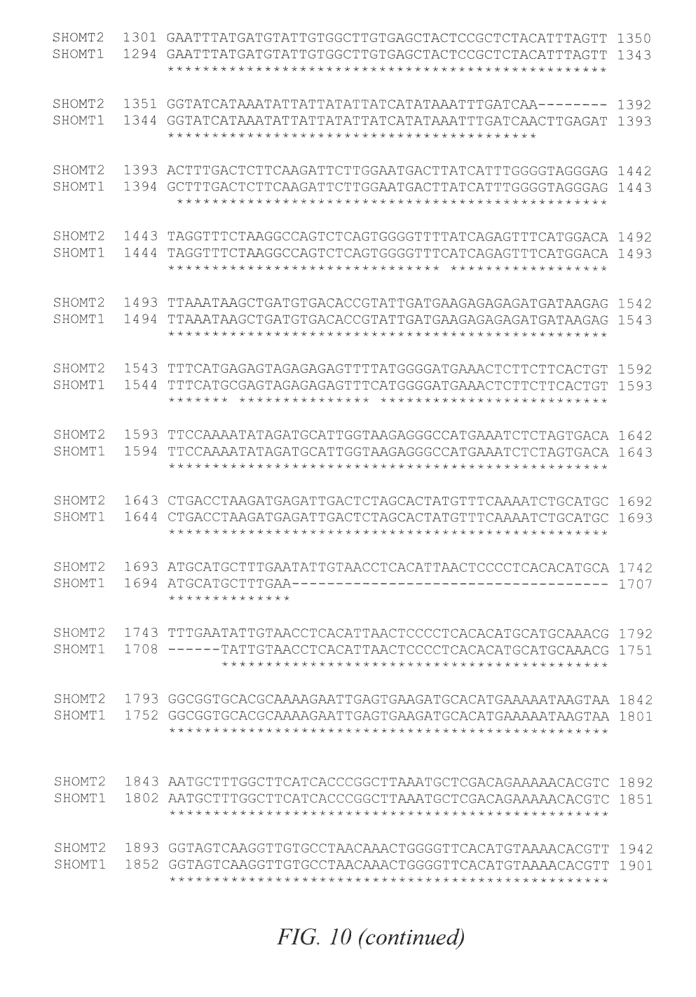

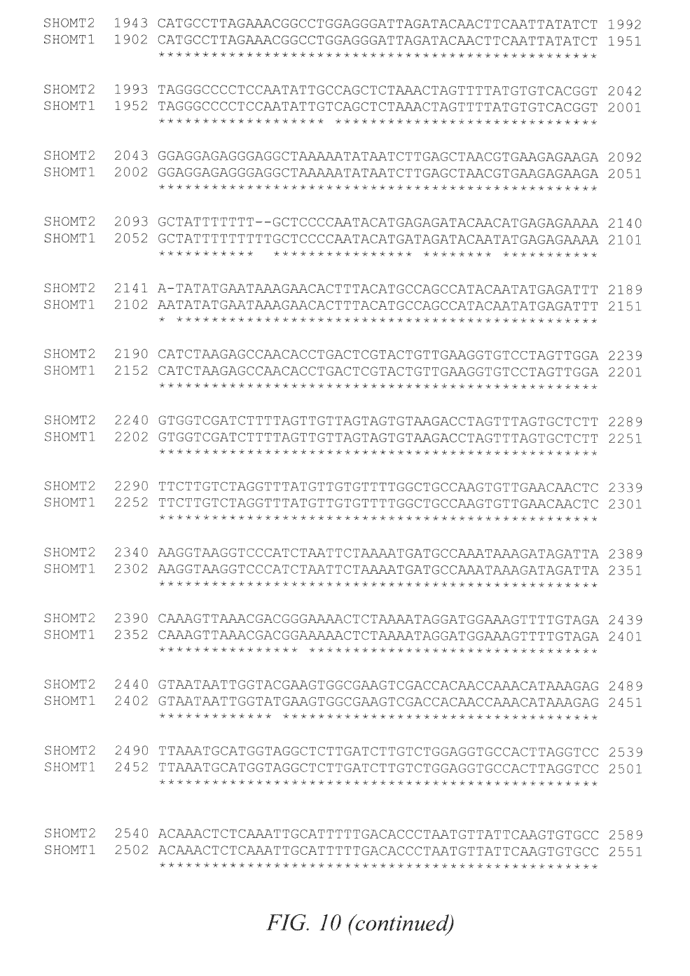

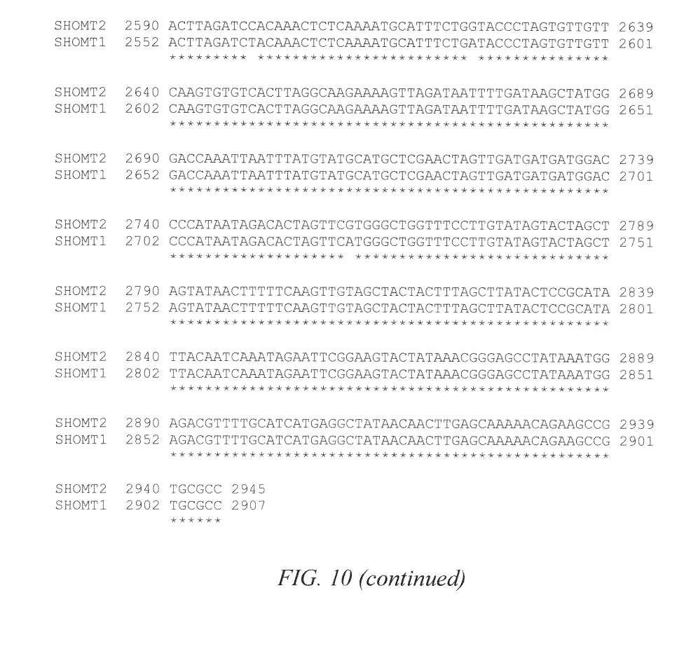

In some embodiments, an expression control sequence may comprise one or more promoters, one or more operators, one or more enhancers, one or more ribosome binding sites, and/or combinations thereof. An expression control sequence may comprise, for example, a nucleic acid (a) operable to direct stem-regulated and/or defense-inducible expression in one or more monocots including monocot crops (e.g., sugarcane, sorghum, maize, and rice) and (b) having a nucleotide sequence more than about 70% identical to SEQ ID NO: 1, more than about 75% identical to SEQ ID NO: 1, more than about 80% identical to SEQ ID NO: 1, more than about 81% identical to SEQ ID NO: 1, more than about 82% identical to SEQ ID NO: 1, more than about 83% identical to SEQ ID NO: 1, more than about 84% identical to SEQ ID NO: 1, more than about 85% identical to SEQ ID NO: 1, more than about 86% identical to SEQ ID NO: 1, more than about 87% identical to SEQ ID NO: 1, more than about 88% identical to SEQ ID NO: 1, more than about 89% identical to SEQ ID NO: 1, more than about 90% identical to SEQ ID NO: 1, more than about 92% identical to SEQ ID NO: 1, more than about 94% identical to SEQ ID NO: 1, more than about 96% identical to SEQ ID NO: 1, more than about 98% identical to SEQ ID NO: 1, more than about 98.5% identical to SEQ ID NO: 1, more than about 99% identical to SEQ ID NO: 1, and/or more than about 99.5% identical (e.g., 100% identical) to SEQ ID NO: 1. For example, an isolated nucleic acid may comprise an expression control sequence (e.g., promoter) isolated from sugarcane having the sequence of nucleotides −4726 to −1 as depicted in FIG. 1 (nucleotides 1 to 4726 of SEQ ID NO:1). According to some embodiments, sequences that are not 100% identical over the full length of SEQ ID NO: 1 may have points and/or regions of variation that are dispersed (e.g., uniformly, haphazardly, randomly) over the length of the subject nucleic acid. For example, an expression control sequence may comprise one or more regions of sequence that are 100% identical to SEQ ID NO: 1 (e.g., in or near a TATA-box, a CCAAT-box, a TSS-motif, and/or one or more of the motifs in Table 2) and one or more regions that are less than 100% identical length and/or sequence. An expression control sequence in some embodiments, may comprise a nucleic acid having a nucleotide sequence that is about 100% identical to a consensus sequence of SHOMT1 (SEQ ID NO: 4) and nucleotides 1-4726 of SHOMT2 (SEQ ID NO: 1) (e.g., FIG. 10). In some embodiments, a consensus sequence (with or without gaps) may be generated using algorithms such as MULTALIN and/or CLUSTALW and full length (or fragments over about 2.9 kb) of SEQ ID NOS: 1 and 4. An expression control sequence may comprise a nucleic acid having a nucleotide sequence that is more than about 95% identical to SEQ ID NO: 1 over the remaining (non-consensus) sequences according to some embodiments. Nucleotides at non-consensus sequence positions may be selected from the nucleotide at that position in SEQ ID NO: 1, the nucleotide at that position in SEQ ID NO: 4, and/or another nucleotide. An expression control sequence may comprise, in some embodiments, a nucleic acid having less than 98% (e.g., less than 97.9%, less than about 97.5%, less than about 97%) identical to SEQ ID NO: 4 over its length. An expression control sequence in some embodiments, may comprise a nucleic acid having a nucleotide sequence that is about 100% identical to a consensus sequence of SHOMT1 (SEQ ID NO: 4) and SHOMT2 (SEQ ID NO: 1) (e.g., SEQ ID NO: 5), more than about 95% identical to SEQ ID NO: 1 over the remaining (non-consensus) sequences (e.g., 2946-4726), and/or less than about 98% (e.g., less than about 97.5%, less than about 97%) identical over its length to SEQ ID NO: 4.

According to some embodiments, an expression control sequence may comprise, for example, a nucleic acid having a nucleic acid sequence at least about 98% identical to nucleotides 1-4726 of SEQ ID NO: 1 or a nucleic acid having a nucleic acid sequence at least about 98% identical to nucleotides 1-4679 of SEQ ID NO: 1 (e.g., without the 5′UTR). An expression control sequence may comprise a nucleic acid having nucleic acid sequence at least about 98% identical to nucleotides 1-2969 of SEQ ID NO: 4 (e.g., without the 5′UTR) in some embodiments.

It will be understood by one skilled in the art that where the designation “SHOMT2 promoter” is used in the present description, use of other nucleic acids having similar hybridization characteristics, expression characteristics, and/or sequence identity, as set forth herein may be substituted. According to some embodiments, expression control sequences (e.g., less than 100% identical to SEQ ID NO:1) retain some ability to direct stem-specific transcription and/or defense-inducible transcription in at least one monocot (e.g., sugarcane, sorghum, maize, rice).

A number of algorithms, often implemented on a computer, are available to compare and align nucleic acid sequences which one skilled in the art may use for purposes of determining sequence identity (sequence similarity) including, for example, the Basic Local Alignment Search Tool (BLAST), ClustalW, ClustalX, FASTA, LALIGN, GGSEARCH, and/or GLSEARCH. For example, sequences similar to a subject expression control sequence (e.g., promoter) may be identified, according to some embodiments, by database searches using the expression control sequence (e.g., promoter) or elements thereof as the query sequence with a sequence search/alignment algorithm (e.g., the Gapped BLAST algorithm (Altschul et al., 1997 Nucl. Acids Res. 25:3389-3402) with the BLOSUM62 Matrix, a gap cost of 11 and persistence cost of 1 per residue and an E value of 10.) Two sequences may be compared with either ALIGN (Global alignment) or LALIGN (Local homology alignment) in the FASTA suite of applications (Pearson and Lipman, 1988 Proc. Nat. Acad. Sci. 85:2444-24448; Pearson, 1990 Methods in Enzymology 183:63-98) with the BLOSUM50 matrix and gap penalties of −16, −4.

A nucleic acid comprising an expression control sequence, in some embodiments, may hybridize with the SHOMT2 nucleic acid sequence as set forth in FIG. 1 (SEQ ID NO:1), may differ in one or more positions in comparison with SEQ ID NO:1, and/or may be operable to direct stem-regulated and/or defense-inducible expression in at least one monocot. Hybridization may include conventional nucleic acid hybridization conditions, which may be stringent. Stringent hybridization conditions may include, for example, (a) hybridization in 4×SSC at 65° C., followed by washing in 0.1×SSC at 65° C. for one hour and/or (b) hybridization in 50% formamide, 4×SSC at 42° C.

In some embodiments, stem-specificity and/or defense-inducibility of an expression control sequence may be confirmed by constructing transcriptional and/or translational fusions of a test sequence with a coding sequence of a heterologous gene and/or coding sequence, transfering the resulting fusion (e.g., in an expression cassette) into an appropriate host, and detecting expression of the heterologous gene and/or coding sequence. The detected expression may be compared to a corresponding fusion with SEQ ID NO:1 and/or a modified version thereof. The assay used to detect expression depends upon the nature of the heterologous gene and/or coding sequence. For example, reporter genes (e.g., chloramphenicol acetyl transferase, β-glucuronidase (GUS), fluorescent protein) may be used to assess transcriptional and translational competence of chimeric nucleic acids. Standard assays are available to sensitively detect reporter enzymes in a transgenic organism.

The GUS gene is useful as a reporter of expression control sequence (e.g., promoter) activity in transgenic plants because of the high stability of the enzyme in plant cells, the lack of intrinsic GUS activity in higher plants, and availability of a quantitative fluorimetric assay and a histochemical localization technique. Jefferson et al. (EMBO Journal 6:3901-3907, 1987) have established standard procedures for biochemical and histochemical detection of GUS activity in plant tissues. Biochemical assays may be performed by mixing plant tissue lysates with 4-methylumbelliferyl-β-D-glucuronide, a fluorimetric substrate for GUS, incubating one hour at 37° C., and then measuring the fluorescence of the resulting 4-methyl-umbelliferone. Histochemical localization for GUS activity is determined by incubating plant tissue samples in 5-bromo-4-chloro-3-indolyl-glucuronide (X-Gluc) for about 18 hours at 37° C. and observing the staining pattern of X-Gluc. Construction of such expression cassettes may allow definition of specific regulatory sequences and may demonstrate that a test sequence can direct expression of heterologous genes, and/or coding sequences in a stem-regulated and/or defense-inducible manner.

### Expression Cassettes and Vectors

The disclosure relates, in some embodiments, to expression vectors and/or expression cassettes for expressing a nucleic acid sequence (e.g., a coding sequence) in a cell and comprising an expression control sequence and the nucleic acid sequence operably linked to the expression control sequence. A cassette, in some embodiments, may include a nucleotide sequence capable of expressing a particular coding sequence inserted so as to be operably linked to one or more expression control sequences present in the nucleotide sequence. Thus, for example, an expression cassette may include a heterologous coding sequence which is desired to be expressed in one or more plant cells, plant tissues, and/or one or more plant organs up to and including a whole plant, according to some embodiments. In some embodiments, an expression cassette may comprise an expression control sequence operable to direct stem-regulated and/or defense-inducible expression of a nucleic acid sequence (e.g., a coding sequence).

An expression control sequence (e.g., promoter), according to some embodiments, may be useful in the construction of an expression cassette comprising, in a 5′ to 3′ direction, the expression control sequence (e.g., SHOMT2), a nucleic acid having a desired sequence for expression (e.g., a coding sequence, an antisense sequence, a heterologous gene), and/or sequence complementary to a native plant gene (e.g., under control of the expression control sequence), and/or a 3′ termination sequence. In some embodiments, an expression cassette may be operable to facilitate and/or drive expression of a nucleic acid having a desired sequence (e.g., a bioinsecticidal peptide and/or a defense elicitor peptide) for expression in a stem-regulated and/or defense-inducible manner. According to some embodiments, an expression cassette may comprise, in a 5′ to 3′ direction, two or more expression control sequences (e.g., tandem copies of SHOMT2, SHOMT2 in tandem with another expression control sequence, another expression control sequence in tandem with SHOMT2), a nucleic acid having a desired sequence for expression, and (optionally) one or more termination sequences.

An expression cassette may be constructed by ligating an expression control sequence (e.g., SHOMT2 and/or a portion thereof) to a coding sequence of a heterologous gene. Juxtaposition of these sequences may be accomplished in a variety of ways. In one embodiment, the sequences may be ordered in a 5′ to 3′ direction expression control sequence, desired sequence for expression, and optionally, a termination sequence (e.g., including a polyadenylation site).

An expression cassette may be incorporated into a variety of autonomously replicating vectors in order to construct an expression vector according to some embodiments. Standard techniques known to those of ordinary skill in the art for construction of an expression cassette may be used. A variety of strategies are available for ligating fragments of DNA, the choice of which depends on the nature of the termini of the DNA fragments.

Restriction and/or deletion fragments that contain an expression control sequence (e.g., promoter) TATA box may be ligated, according to some embodiments, in a forward orientation to a promoterless heterologous gene and/or a coding sequence, for example, a coding sequence of GUS. In some embodiments, an expression control sequence (e.g., promoter) may be prepared, for example, by chemical and/or enzymatic synthesis.

A 3′ end of a heterologous coding sequence may be optionally ligated to a termination sequence including a polyadenylation site (e.g., a nopaline synthase polyadenylation site, and/or an octopine T-DNA gene 7 polyadenylation site). Alternatively, a polyadenylation site may be included in a heterologous gene and/or a coding sequence.

According to some embodiments, the disclosure relates to an expression cassette, which may comprise, for example, a nucleic acid having an expression control sequence and a coding sequence operably linked to the expression control sequence. An expression cassette may be comprised in an expression vector. A coding sequence, in some embodiments, may comprise any coding sequence expressible in at least one plant cell. For example, a coding sequence may comprise a human sequence (e.g., an antibody sequence), a non-human animal sequence, a plant sequence, a yeast sequence, a bacterial sequence, a viral sequence (e.g., plant virus, animal virus, and/or vaccine sequence), an artificial sequence, an antisense sequence thereof, a fragment thereof, a variant thereof, and/or combinations thereof. According to some embodiments, a coding sequence may comprise, a sugar transport gene and/or a sugar accumulation gene. Examples of sugar transport genes may include, without limitation, a disaccharide transporter (e.g., a sucrose transporter) and/or a monosaccharide transporter. A coding sequence may comprise, in some embodiments, a sequence encoding one or more gene products with insecticidal, antimicrobial, and/or antiviral activity. Examples of gene products that may have insecticidal activity, antimicrobial activity, and/or antiviral activity may include, without limitation, avidin, vegetative insecticidal proteins (e.g., Vip3A), insecticidal crystal proteins from Bacillus thuringiensis (e.g., Cry1, Cry1Ab, Cry2, Cry9), pea albumin (e.g., PA1b), hirsutellin A, lectins (e.g., snow drop lily lectin, garlic lectin, onion lectin), amylase inhibitors (e.g., alpha amylase inhibitor), arcelins (e.g., arcelins from beans), proteinase inhibitors, lysozymes (e.g., bovine lysozyme, human lysozyme, mollusk lysozyme), defensin, chitinase, β-1,3-glucanase, variants thereof, and/or combinations thereof. A coding sequence may comprise an enzyme for forming and/or modifying a polymer according to some embodiments. Examples of enzymes for forming and/or modifying a polymer may include, without limitation, a polyhydroxyalkanoate synthases, 4-hydroxybutyryl-CoA transferases, variants thereof, and/or combinations thereof. In some embodiments, a coding sequence may comprise a sequence encoding one or more enzymes that hydrolyzes cellulose. Examples of enzymes that hydrolyze cellulose include, without limitation, cellulase, endoglucanases (e.g., endo β-1,4 glucanases), glucosidases (e.g., βglucosidase), hydrolases (e.g., β-1,4-glucan cellobiohydrolase), exocellulases, variants thereof, and/or combinations thereof. In some embodiments, a coding sequence may comprise a sequence encoding one or more enzymes that form and/or modify a sugar (e.g., sucrose, trehalose, sorbitol, fructan, fructose, tagatose, sucralose). Examples of enzymes that form and/or modify a sugar may include, without limitation, trehalose-6-phosphate synthase (TPS) and trehalose-6-phosphate phosphatase (TPP). According to some embodiments, a coding sequence may comprise a sequence encoding an enzyme for forming or modifying glycine betaine, a polyamine, proline, threhalose, a variant thereof, and/or combinations thereof. A coding sequence may comprise, in some embodiments, a start codon, an intron, and/or a translation termination sequence. According to some embodiments, a coding sequence may comprise one or more natural or artificial coding sequences (e.g., encoding a single protein or a chimera). According to some embodiments, an expression cassette may optionally comprise a termination sequence.

An expression control sequence may be used, in some embodiments, to construct an expression cassette comprising, in the 5′ to 3′ direction, (a) the expression control sequence (e.g., a SHOMT2 promoter), (b) a heterologous gene or a coding sequence, or sequence complementary to a native plant gene under control of the expression control sequence, and/or (c) a 3′ termination sequence (e.g., a termination sequence comprising a polyadenylation site). Examples of expression cassettes may include, in some embodiments, SEQ ID NO: 2 and/or SEQ ID NO:3. An expression cassette may be incorporated into a variety of autonomously replicating vectors in order to construct an expression vector. An expression cassette may be constructed, for example, by ligating an expression control sequence to a sequence to be expressed (e.g., a coding sequence).

Some techniques for construction of expression cassettes are well known to those of ordinary skill in the art. For example, a variety of strategies are available for ligating fragments of DNA, the choice of which depends on the nature of the termini of the DNA fragments. Restriction and/or deletion fragments that contain a subject promoter TATA box may be ligated in a forward orientation to a promoterless heterologous gene or coding sequence such as the coding sequence of GUS. An artisan of ordinary skill having the benefit of the present disclosure, an expression control sequence and/or portions thereof may be provided by other means, for example chemical or enzymatic synthesis.

A nucleic acid may comprise, in a 5′ to 3′ direction, an expression control sequence, a linker (optional), and a coding sequence according to some embodiments. A linker may be, in some embodiments, from about 1 nucleotide to about 200 nucleotides in length and/or may comprise one or more restriction sites. Expression level of a nucleic acid sequence (e.g., a coding sequence) operably linked to an expression control sequence may be influenced by the length and/or sequence of a linker and/or the 5′ sequence of the coding sequence. For example, expression level may be influenced by the sequence from about the −4 position to about the +4 position. In some embodiments, a nucleic acid may comprise, in a 5′ to 3′ direction, an expression control sequence, a linker, and a coding sequence, wherein the sequence of positions −4 to +4 comprises a sequence selected from the sequence shown in Table 1. A nucleic acid may comprise, in a 5′ to 3′ direction, an expression control sequence and a coding sequence, wherein the sequence of positions −4 to +4 comprises a sequence selected from the sequence shown in Table 1 according to some embodiments. In some embodiments, a −3 to −1 sequence of AAA may be associated with higher (e.g., the highest) expression levels than other −3 to −1 sequences. A +1 to +4 sequence of ATGG may be associated with higher (e.g., the highest) expression levels than other +1 to +4 sequences (e.g., ATGC, ATGA, ATGT).

TABLE 1
Optional Junction Sequences

| | −4 | −3 | −2 | −1 | +1 | +2 | +3 | +4 |
|---|---|---|---|---|---|---|---|---|
| 1 | N | N | N | N | A | T | G | G/T |
| 2 | N | A/C | A/C | A/C | A | T | G | G |
| 3 | A/C | A/C | A/C | A/C | A | T | G | G |
| 4 | N | A | A | A | A | T | G | G |
| 5 | N | A | A | C | A | T | G | G |
| 6 | N | A | C | A | A | T | G | G |
| 7 | N | A | C | C | A | T | G | G |
| 8 | N | C | A | A | A | T | G | G |
| 9 | N | C | A | C | A | T | G | G |
| 10 | N | C | C | A | A | T | G | G |
| 11 | N | C | C | C | A | T | G | G |
| 12 | N | A | A | T | A | T | G | G |
| 13 | N | A | T | A | A | T | G | G |
| 14 | N | A | T | T | A | T | G | G |
| 15 | N | T | A | A | A | T | G | G |
| 16 | N | T | A | T | A | T | G | G |
| 17 | N | T | T | A | A | T | G | G |
| 18 | N | T | T | T | A | T | G | G |
| 19 | N | C | T | T | A | T | G | G |
| 20 | N | T | C | T | A | T | G | G |
| 21 | N | T | T | C | A | T | G | G |
| 22 | C | A | C | C | A | T | G | G |
| 23 | N | N | C | C | A | T | G | G |
| 24 | C | G | C | C | A | T | G | G |
| 25 | N | A/C | A/C | A/C | A | T | G | G |
| 26 | A/C | A/C | A/C | A/C | A | T | G | G |
| 27 | N | A | A | A | A | T | G | G |
| 28 | N | A | A | C | A | T | G | G |
| 29 | N | A | C | A | A | T | G | G |
| 30 | N | A | C | C | A | T | G | G |
| 31 | N | C | A | A | A | T | G | G |
| 32 | N | C | A | C | A | T | G | G |
| 33 | N | C | C | A | A | T | G | G |
| 34 | N | C | C | C | A | T | G | G |
| 35 | N | A | A | T | A | T | G | G |
| 36 | N | A | T | A | A | T | G | G |
| 37 | N | A | T | T | A | T | G | G |
| 38 | N | T | A | A | A | T | G | G |
| 39 | N | T | A | T | A | T | G | G |
| 40 | N | T | T | A | A | T | G | G |
| 41 | N | T | T | T | A | T | G | G |
| 42 | N | C | T | T | A | T | G | G |
| 43 | N | T | C | T | A | T | G | G |
| 44 | N | T | T | C | A | T | G | G |
| 45 | C | A | C | C | A | T | G | G |
| 46 | N | N | C | C | A | T | G | G |
| 47 | C | G | C | C | A | T | G | G |

In some embodiments, the 3′ end of a heterologous coding sequence may be operably linked to a termination sequence including, for example, a polyadenylation site, exemplified by, but not limited to, a nopaline synthase polyadenylation site and/or a octopine T-DNA gene 7 polyadenylation site. A polyadenylation site may be provided by the heterologous gene or coding sequence according to some embodiments.

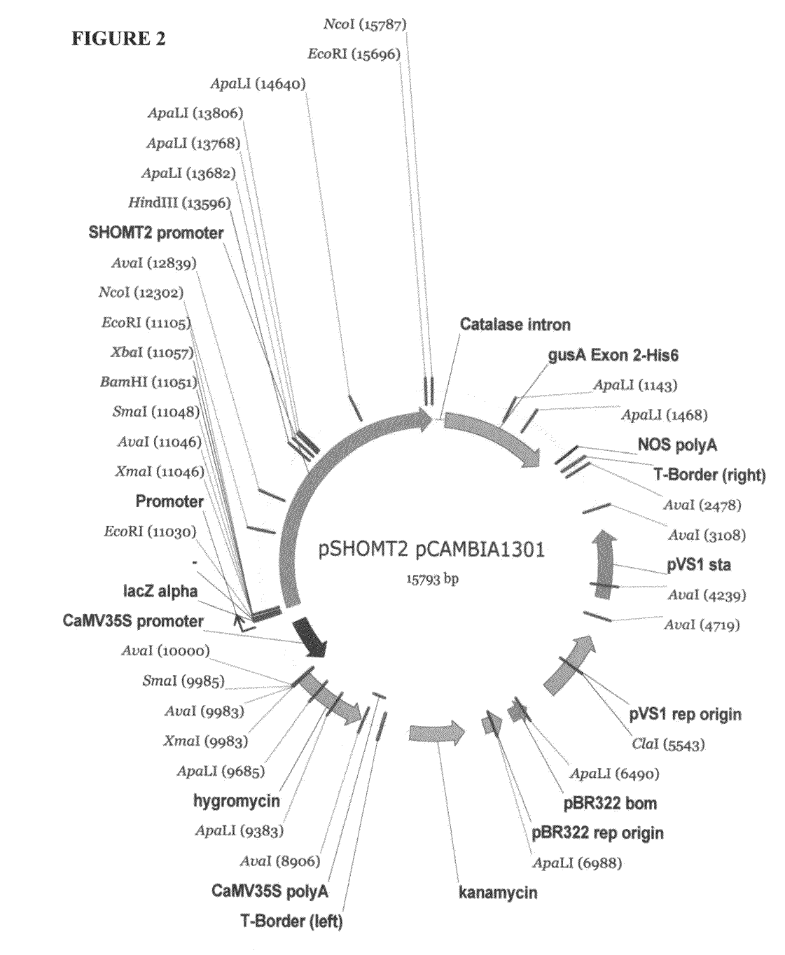

The present disclosure relates, in some embodiments, to expression vectors including a nucleic acid having an expression control sequence operable to direct stem-regulated and/or defense-inducible expression. An expression vector may comprise, for example, a nucleic acid having an expression control sequence and a coding sequence operably linked to the expression control sequence. An expression vector may be contacted with (e.g., transferred into) a cell (e.g., a plant cell) in such a manner as to allow expression (e.g., transcription) of an expression vector-encoded gene product (e.g., protein) in the cell and/or one or more tissues derived from the cell. An expression control sequence may be contacted with a plant cell (e.g., an embryonic cell, a stem cell, a callus cell) under conditions that permit expression of the coding sequence in the cell and/or cells derived from the plant cell according to some embodiments. A vector may be transmitted into a plant cell in such a manner as to allow inheritance of the nucleic acid into daughter cells (e.g., somatic cells, gametes). For example, a nucleic acid may be inherited by the second progeny of plants generated from a plant derived from the transformed plant cell. In some embodiments, such inheritance may be Mendelian. Examples of expression vectors may include, without limitation the vectors shown in FIG. 1 and FIG. 2. According to some embodiments, an expression vector may include one or more selectable markers. For example, an expression vector may include a marker for selection when the vector is in a bacterial host, a yeast host, and/or a plant host.

According to some embodiments, an expression control sequence (e.g., to be contacted with a target cell) may be included in an expression cassette and/or an expression vector. In some embodiments, an expression control sequence may be included in a plant transformation vector (e.g., a binary vector). A binary vector may comprise native and/or modified portions of Agrobacterium tumefaciens T-DNA in some embodiments.

### Microorganisms

The present disclosure relates, in some embodiments, to a microorganism comprising an expression control sequence. For example, a microorganism may comprise a bacterium, a yeast, and/or a virus. In some embodiments, an expression control sequence may comprise an expression control sequence (e.g., promoter), which directs stem-regulated and/or defense-inducible expression (e.g., a SHOMT2 promoter). A microorganism may comprise an expression control sequence and a coding sequence operably linked to the expression control sequence. Examples of microorganisms may include, without limitation, Agrobacterium tumefaciens, Escherichia coli, a lepidopteran cell line, a Rice tungro bacilliform virus, a Commelina yellow mosaic virus, a Banana streak virus, a Taro bacilliform virus, and/or baculovirus. An expression control sequence may be present on a genomic nucleic acid and/or an extra-genomic nucleic acid.

### Plants

The present disclosure relates, in some embodiments, to a plant cell (e.g., an embryonic cell, a stem cell, a callus cell), a tissue, and/or a plant comprising an expression control sequence. A plant and/or plant cell may be a monocot cell (e.g., maize, rice, sugarcane and/or sorghum) in some embodiments. Examples of a monocot may include, without limitation, sugarcane, miscanthus, a miscanthus×sugarcane hybrid, switch grass, oat, wheat, barley, maize, rice, banana, yucca, onion, asparagus, and/or sorghum. A plant cell may be included in a plant tissue, a plant organ, and/or a whole plant in some embodiments. A plant cell in a tissue, organ, and/or whole plant may be adjacent, according to some embodiments, to one or more isogenic cells and/or one or more heterogenic cells. In some embodiments, a plant may include primary transformants and/or progeny thereof. A plant comprising an expression control sequence may further comprise a transgene operably linked to the expression control sequence, in some embodiments. A transgene may be expressed, according to some embodiments, in a plant comprising an expression control sequence in all (e.g., substantially all) organs, tissues, and/or cell types including, without limitation, stalks, leaves, roots, seeds, flowers, fruit, meristem, parenchyma, storage parenchyma, collenchyma, sclerenchyma, epidermis, mesophyll, bundle sheath, guard cells, protoxylem, metaxylem, phloem, phloem companion, and/or combinations thereof. A transgene operably linked to an expression control sequence, according to some embodiments, may display stem-regulated and/or defense-inducible expression. In some embodiments, a transgene and/or its gene product may be located in and/or translocated to one or more organelles (e.g., vacuoles, chloroplasts, mitochondria, plastids). An expression control sequence may be present on a genomic nucleic acid and/or an extra-genomic nucleic acid. An expression control sequence in a plant cell may be positioned within an expression cassette and/or an expression vector in some embodiments.

### Expression Systems

The present disclosure relates, according to some embodiments, to a system for expression of (e.g., to high levels) of a nucleic acid sequence (e.g., comprising one or more coding sequences). For example, an expression system may be comprised in plants to be used as a biofactory for high-value proteins. Without being limited to any particular mechanism of action, an expression system may benefit from additive and/or synergistic expression control sequence activities, transcriptional synergism, and/or reduced silencing of an introduced coding sequence (e.g., transgene), a phenomenon frequently observed in plants when the same promoters are used to express the same or different transgenes, and constituting a major risk for the economic exploitation of plants as biofactories. Plants comprising an expression system may retain desirable (e.g., high) expression levels through one or more consecutive generations of transgenic plants.

In some embodiments, an expression system may comprise two or more expression control sequences (e.g., promoters) each operably linked to a respective number of clones of a single coding sequence. According to some embodiments, two, three, four, five, or more expression control sequences (e.g., promoters) may be operably linked to two, three, four, five, or more clones of a single coding sequence. Each expression control sequence independently may be constitutive and/or regulated (e.g., tissue-specific expression, developmentally-inducible expression, stress-inducible expression, defense-inducible expression, and/or drought-inducible expression) according to some embodiments. In some embodiments, each clone of a coding sequence may be identical to one or more of the other clones. Copies of a coding sequence, according to some embodiments, may differ from one another somewhat, for example, where one copy may be codon optimized for one family, genus, and/or species while another may be optimized for a different family, genus, and/or species, or not codon optimized at all. Each expression control sequence-coding sequence clone independently may be present (e.g., in a microorganism and/or plant) on an expression vector, on a genomic nucleic acid, and/or on an extra-genomic nucleic acid in some embodiments. Each expression control sequence-coding sequence clone independently, in some embodiments, may further comprise one or more terminators.

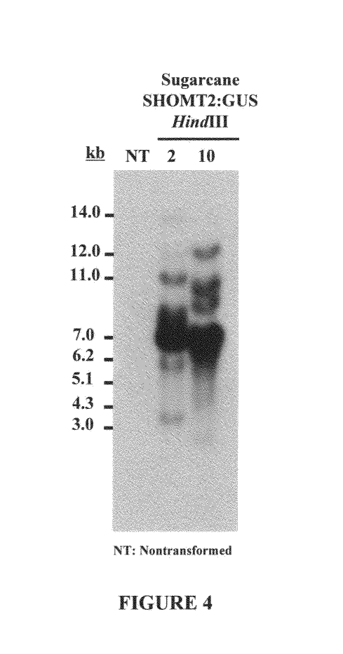

The present disclosure relates, according to some embodiments, to transgenic plants of sugarcane, a high biomass producer and sugar accumulator, which are generated from embryonic callus transformed with an expression vector (e.g., comprising a SHOMT2 promoter and a β-glucuronidase (GUS) reporter gene). For example, SHOMT2:GUS transgenic sugarcane plants according to some embodiments of the disclosure were observed expressing high levels of GUS driven by the SHOMT2 promoter in the stem (e.g., SHOMT2 confers stem-regulated gene expression), up to 128.2 pmoles of 4-methylumbelliferone /min/μg total protein, with 28% of the expression found in the stem nodes. The stems were noted to show maximal increases in GUS expression of 7.5-fold and 2.3-fold compared to leaves and roots, respectively. The SHOMT2:GUS transgenic sugarcane plants according to some embodiments of the disclosure were observed histochemically to express the GUS gene driven by the SHOMT2 promoter in the stem vascular bundles (e.g., SHOMT2 confers vascular gene expression), preferentially in the phloem companion cells and surrounding bundle sheath cells of the schlerenchymatous tissue (FIG. 4), and in the storage parenchyma (FIG. 4).

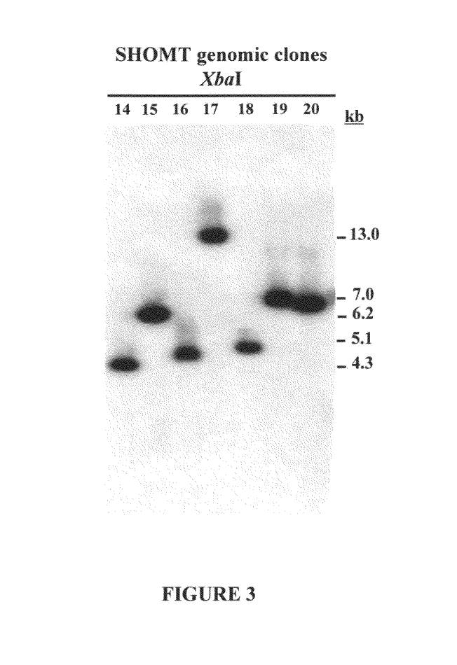

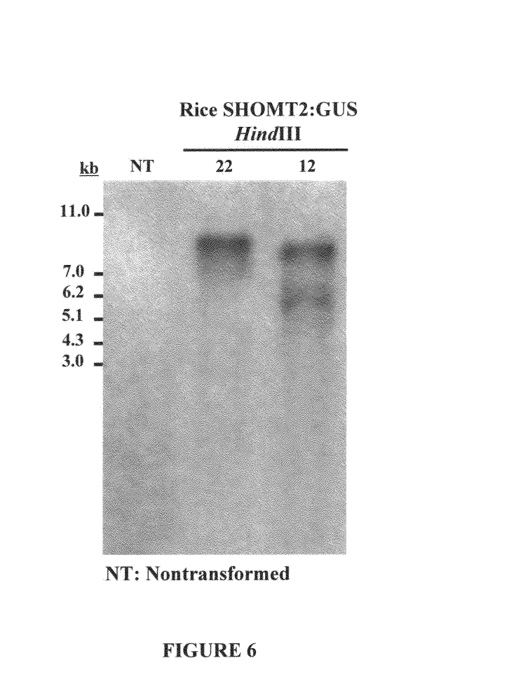

The present disclosure relates, according to some embodiments, to transgenic plants of rice, an important staple food crop, which are generated from embryogenic callus transformed with the expression vector, SHOMT2 promoter and β-glucuronidase (GUS) reporter gene (e.g., one promoter-one transgene system) (FIG. 3) (SEQ ID NO: 3). The rice SHOMT2:GUS transgenic plants according to some embodiments of the disclosure were observed expressing high levels of GUS driven by the SHOMT2 promoter in the culm (e.g., SHOMT2 confers culm-regulated expression), up to 301.8 pmoles of 4-methylumbelliferone /min/μg total protein. The stems were noted to show maximal increases in GUS expression of 215.6-fold and 5.3-fold compared to leaves and roots, respectively. The SHOMT2:GUS transgenic rice plants according to some embodiments of the disclosure were observed histochemically to express the GUS gene driven by the SHOMT2 promoter in the stem vascular bundles (e.g., SHOMT2 confers vascular gene expression), mainly in the vascular parenchymatous tissue surrounding the xylem (FIG. 6), and in the storage parenchyma (FIG. 6).

The present disclosure relates, in some embodiments, to methods for producing one promoter-one transgene expression vectors and the transgenic plants. Methods may be used, for example, to transform different varieties of sugarcane or rice by co-bombarding or co-cultivating a target explant tissue (e.g., embryogenic callus or leaf roll disc) with a transgene (e.g., a β-glucuronidase reporter gene) under the control of an expression control sequence (e.g., SHOMT2 promoter).

### Methods

According to some embodiments, the present disclosure relates to methods for transforming and/or transfecting a plant with a nucleic acid comprising an expression control sequence. For example, a method may comprise contacting a cell (e.g., a yeast cell and/or a plant cell) with a nucleic acid comprising an expression control sequence. Contacting a nucleic acid with a cell may comprise, in some embodiments, co-cultivating a target cell with a bacterium (e.g., Agrobacterium) comprising the nucleic acid (e.g., in a binary vector), electroporating a cell in the presence of the nucleic acid, infecting a cell with a virus (baculovirus) comprising the nucleic acid, bombarding a cell (e.g., a cell in a leaf, stem, and/or callus) with particles comprising the nucleic acid, agitating a cell in a solution comprising the nucleic acid and one or more whiskers (e.g., silicone carbide whiskers), and/or chemically inducing a cell to take up extracellular DNA. In some embodiments, contacting a nucleic acid with a cell may comprise contacting the nucleic acid with a plant leaf disc and/or a plant protoplast.

For example, embryonic calli and/or and other susceptible tissues may be inoculated with a binary vector comprising an expression control sequence and optionally A. tumefaciens T-DNA, cultured for a number of days, and then transferred to antibiotic-containing medium. Transformed shoots may be then selected after rooting in medium containing the appropriate antibiotic, and transferred to soil. Transgenic plants may be pollinated and seeds from these plants may be collected and grown on antibiotic medium.

A transgenic plant may comprise, in some embodiments, a monocot (e.g., sugarcane, rice, maize, sorghum). A transgenic line may be maintained from cuttings of a transgenic plant according to some embodiments. For example, a trangenic line having a transgene that is somatically and (optionally) stably inherited may be maintained from cuttings of the original transformant.

Expression of a sequence of interest (e.g., a heterologous gene, a transgene, a reporter gene) in a cell, a tissue, a seed (e.g., a developing seed), a tissue, a young seedling and/or a mature plant may be detected and/or monitored in some embodiments. For example, expression of a sequence of interest may be monitored and/or detected by one or more immunological assays, one or more histochemical assays, one or more mRNA expression assays, one or more activity (e.g., catalytic activity) assays, and/or combinations thereof. According to some embodiments, the choice of an assay may be influenced by and/or depend upon the nature of the sequence of interest. For example, RNA gel blot analysis may be used to assess transcription where appropriate nucleotide probes are available. Where antibodies to the polypeptide encoded by a sequence of interest are available, western analysis and immunohistochemical localization may be used to assess the production and/or localization of an encoded polypeptide. Where a sequence of interest encodes a gene product with catalytic activity and/or detectable biochemical properties, appropriate biochemical assays may be used.

The disclosure relates, in some embodiments, to methods for expressing a nucleic acid sequence (e.g., comprising one or more coding sequences) in a cell. For example, a method may comprise contacting a cell (e.g., a yeast cell and/or a plant cell) with a nucleic acid comprising an expression control sequence and a coding sequence operably linked to the expression control sequence under conditions that permit expression of the coding sequence. Expression, according to some embodiments, may be constitutive, conditional, native (e.g., in the normal time and/or tissue), and/or ectopic. In some embodiments, a method may further comprise expressing a nucleic acid sequence in a plant (e.g., a monocot). A method may include harvesting (e.g., partially purifying) from a plant a gene product of a nucleic acid sequence (e.g., an exogenous sequence) expressed in the plant, according to some embodiments. The disclosure relates, in some embodiments, to methods for directing stem-regulated expression and/or defense-inducible expression in a tissue and/or plant. A method may include, for example, providing a tissue and/or plant with an isolated nucleic acid having an expression control sequence (e.g., a SHOMT2 promoter) to effect such stem-regulated and/or defense-inducible expression.

In some embodiments, the present disclosure relates to methods for isolating an expression control sequence operable in at least one monocot. For example, a method may comprise screening a library (e.g., a plant genomic library, a bacterial artificial chromosome library, a plant virus genomic library) with a probe comprising a nucleic acid having a nucleic acid sequence of SEQ ID NO: 1, a complement thereof, and/or a portion thereof (e.g., under stringent hybridization conditions). A method may comprise amplifying an expression control sequence from a library (e.g., using a polymerase chain reaction) using one or more primers derived from a nucleic acid sequence of SEQ ID NO: 1, a complement thereof, and/or a portion thereof. Operability of a candidate expression control sequence in at least one monocot may be confirmed, in some embodiments, by forming a transcriptional and/or translational fusion of a candidate expression control sequence with a coding sequence expressible in the at least one monocot to form an expression cassette, transferring the expression cassette into the at least one monocot, and/or detecting expression of the coding sequence. An assay for detecting expression of the coding sequence may depend on the nature of the coding sequence. For example, a coding sequence may comprise a reporter gene (e.g., an autofluorescent protein, chloramphenicol acetyl transferase, β-glucuronidase (GUS)). Standard assays are available to sensitively detect a reporter enzyme in a transgenic organism.

The present disclosure relates, according to some embodiments, to methods for isolating an expression control sequence operable in at least one monocot. For example, a method may comprise selecting one or more primers from about 15 to about 40 nucleotides in length and corresponding to (but not necessarily identical to) sequences at or near the 5′ and/or 3′ ends of SEQ ID NO: 1, contacting the one or more primers with an amplification library (e.g., a partial or complete viral genomic library, a partial or complete plant genomic library) and a nucleic acid polymerase under conditions that permit amplification of an expression control sequence. A plant genomic library, according to some embodiments, may comprise nucleic acids isolated from a microorganism-infected plant, a microorganism-free plant, a mechanically-injured plant, and/or an injury-free plant. In some embodiments, a method may comprise screening a library with a probe comprising SEQ ID NO:1 or a fragment thereof. One or more candidate expression control sequences (e.g., amplification products) may be cloned into an expression vector in a position to drive expression of a coding sequence (e.g., GUS, an autofluorescent protein). Operability of the amplification products may be assessed, for example, by contacting a plant cell with such expression vectors under conditions that permit expression of the coding sequence (e.g., microprojectile bombardment, Agrobacterium-mediated transformation) and examining the plant cell for the appearance of a gene product of the coding sequence (e.g., the encoded protein).

The present disclosure, in some embodiments, relates to methods of increasing expression levels of a coding sequence in at least one monocot. For example, an expression cassette and/or expression vector may be introduced into a plant in order to effect expression of a coding sequence. According to some embodiments, a method of producing a plant with increased levels of a product of a sucrose accumulating gene and/or a defense gene may comprise transforming a plant cell with an expression vector and/or expression cassette comprising an expression control sequence operably linked to a sucrose accumulating gene or a defense gene and regenerating a plant with increased levels of the product of the sucrose accumulating gene or defense gene. In some embodiments of the present disclosure, a transgenic sugarcane line may be produced in which sugar metabolism is altered to increase stem dry weight (e.g., more than about 50% sucrose, more than about 60% sucrose, more than about 70% sucrose). A transgenic sugarcane line may be produced, according to some embodiments, with enhanced bioinsecticidal activity (e.g., for protection against stem borer insects, which may be the most destructive pests). In some embodiments, expression of a bioinsecticidal protein may be induced by a defense-inducing agent (e.g., salicylic acid, jasmonic acid, methyl jasmonate).

The present disclosure, in some embodiments, relates to methods of decreasing expression levels of a coding sequence (e.g., a native plant sequence, a viral sequence) in at least one monocot. For example, a method may comprise contacting at least one monocot cell with an expression vector comprising an expression control sequence and an antisense sequence that is complementary to at least a portion of the coding sequence and operably linked to the expression control sequence. In some embodiments, a method may comprise contacting at least one monocot cell with an RNA interference (RNAi) expression vector comprising an expression control sequence and a nucleic acid sequence which is an inverted repeat of the native plant gene, the expression level of which is to be reduced and/or silenced, and operably linked to the expression control sequence. A method may comprise, in some embodiments, contacting at least one monocot cell with a cosuppression expression vector comprising an expression control sequence and a nucleic acid sequence coding for the native plant gene operably linked to the expression control sequence.

The present disclosure further relates to methods for isolating and/or purifying (“purifying”) a gene product (e.g., a nucleic acid and/or a protein) from a plant. For example, a method may comprise providing a plant comprising a nucleic acid having an expression control sequence and a coding sequence operably linked to the expression control sequence, wherein the coding sequence encodes a gene product of interest. A method may comprise, according to some embodiments, producing a transgenic protein in a plant, extracting juice containing the transgenic protein from the plant, cleaning the juice to remove particulate matter, and/or transmitting the juice through at least one membrane to produce two fractions, one of the fractions containing the transgenic protein. In some embodiments, a transgenic protein may comprise a lectin, an enzyme, a vaccine, a bacterial lytic peptide, a bacterial lytic protein, an antimicrobial peptide, an antimicrobial peptide protein, an antiviral peptide, an antiviral protein, an insecticidal peptide, an insecticidal protein, a therapeutic peptide, and a therapeutic protein.

As will be understood by those skilled in the art who have the benefit of the instant disclosure, other equivalent or alternative compositions, devices, methods, and systems for expressing a nucleic acid sequence in at least one monocot and/or at least one dicot can be envisioned without departing from the description contained herein. Accordingly, the manner of carrying out the disclosure as shown and described is to be construed as illustrative only.

Persons skilled in the art may make various changes in the shape, size, number, and/or arrangement of parts without departing from the scope of the instant disclosure. For example, the position and number of expression control sequences may be varied. Each disclosed method method step may be performed in association with any other disclosed method or method step and in any order. Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to 50 may include 55, but not 60 or 75. In addition, it may be desirable, in some embodiments, to mix and match range endpoints. Also, in some embodiments, each figure disclosed (e.g., in one or more of the Examples and/or Drawings) may form the basis of a range (e.g., disclosed value +/− about 10%, disclosed value +/− about 100%) and/or a range endpoint. Persons skilled in the art may make various changes in methods of preparing and using a composition, device, and/or system of the disclosure. For example, a composition, device, and/or system may be prepared and or used as appropriate for animal and/or human use (e.g., with regard to sanitary, infectivity, safety, toxicity, biometric, and other considerations).

These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

### EXAMPLES

Some specific example embodiments of the disclosure may be illustrated by one or more of the examples provided herein.

### Example 1

### Isolation of SHOMT2 Genomic Clone and Promoter

The promoter for the sugarcane (Saccharum sp. hybrid) o-methyltransferase 2 gene, SHOMT2 has been isolated by screening a sugarcane genomic library. The nucleic acid sequence of the SHOMT2 promoter has also been determined.

The SHOMT2 genomic clone was isolated from a sugarcane genomic library, constructed in bacteriophage Lambda DASH II vector (Stratagene, Calif.), using standard methods (Crop Science 43:1805-1813, 2003; U.S. Pat. No. 7,323,622). The Lambda genomic library was plated on XL1-Blue MRA bacterial cells, and plaques were transferred to 23 replica nitrocellulose filters (Amersham, N.J.). Replica filters were hybridized using full-length SHOMT cDNA as a probe, according to standard methods (Ausubel et al., Current Protocols in Molecular Biology, 1994). Filters were prehybridized for three hours at 65° C. in hybridization buffer (0.5 M NaHPO₄ pH 7.2, 7% [w/v] SDS, 1 mM EDTA and 1% [w/v] BSA), and hybridized overnight at the same temperature with the SHOMT probe prelabeled radioactively by random priming using Klenow Exo⁻ DNA polymerase (New England Biolabs, Inc., MA). Following hybridization, the filters were washed twice for 15 min each at room temperature with low-stringency wash buffer (40 mM NaHPO₄ pH 7.2, 5% [w/v] SDS, 1 mM EDTA and 0.5% [w/v] BSA), and twice for 20 min each at 65° C. with high-stringency wash buffer (40 mM NaHPO₄ pH 7.2, 1% [w/v] SDS and 1 mM EDTA). The radioactivity signal was detected with an x-ray film after exposure for one day at −95° C. Screening of the bacteriophage genomic library with the SHOMT cDNA probe revealed the presence of several hybridization signals, indicating that the SHOMT gene is present as multiple copies in the sugarcane genome. Seven SHOMT genomic clones exhibiting strong hybridization to the SHOMT cDNA were selected for Southern blot analysis.

The seven Lambda phages (SHOMT genomic clones), which hybridized to the SHOMT cDNA, were plaque purified. Phage DNA was prepared using the liquid lysate protocol (Ausubel et al., Current Protocols in Molecular Biology, 1994), and digested with the restriction endonuclease HindIII at 37° C. for 2 hours and resolved on a 0.6% agarose gel. DNA was transferred by capillary blotting to a Hybond-N^{+™} nylon membrane (Amersham, N.J.) in an alkaline solution (0.4 M sodium hydroxide) (Sambrook and Russell, Molecular Cloning: A laboratory Manual, 2001). DNA gel blot hybridizations using the SHOMT cDNA probe were performed as described for the Lamda genomic library hybridization. Southern blot analysis of the seven SHOMT genomic clones revealed the presence of multiple unique restriction fragments containing the SHOMT gene (FIG. 3), indicating that these SHOMT clones were most likely members of a multigene family.

One SHOMT genomic clone (Clone 15), designated as SHOMT2, was selected for further study (See FIG. 3). A 6.2 kb XbaI fragment of Clone 15 was subcloned into the polylinker site of the pBluescript sequencing vector (Stratagene, Calif.) and sequenced by cycle sequencing using an ABI PRISM dye terminator cycle sequencing kit (Applied Biosystems, CA). The identity of the genomic sequence of the SHOMT2 clone was verified by searching databases through NCBI using the BLASTn algorithm (Altschul et al., Nucleic Acids Research 25:3389-3402, 1997). Genomic and cDNA sequence data for the SHOMT gene was aligned using Sequencher, Version 4.2.2 software (Gene Codes Corp., MI). The SHOMT2 genomic clone was found to contain a 4.726 kb promoter region (upstream regulatory sequence) (SEQ ID NO: 1).

### Example 2

### Comparative Sequence of a SHOMT2 Promoter Relative to Other SHOMT Promoters

The sequence of the SHOMT2 promoter (4.726 kb) (SEQ ID NO: 1) was compared with that of the previously identified SHOMT promoter (2.907 kb). Table 1 shows that the SHOMT2 promoter has 99% identity at −1 to −1209 nucleotides (nt) and 99% identity at −1203 to −2945 nt with the SHOMT promoter.

TABLE 1
Comparison of SHOMT2 and SHOMT promoter sequences

SHOMT (2.907 kb)-GU062719*

| SHOMT2 (4.726 kb) | −1 to −1211 | −1164 to −2907 |
|---|---|---|
| −1 to −1209 nt | 99% | |
| −1203 to −2945 nt | | 99% |

*NCBI GeneBank accession number

Sequence identity (%): The sequence identity (%) was obtained by BLASTn search with the SHOMT2 promoter in NCBI GeneBank

### Example 3

### Identification of Putative Regulatory Motifs Enriched in the SHOMT2 Promoter

The sequence of the SHOMT2 promoter of 4.726 kb (SEQ ID NO: 1) was analyzed with PLACE signal scan (available at http://www.dna.affrc.go.jp/sigscan/signal1.pl), PlantCARE motif sampler (http:// bioinformatics.psb.ugent.be/webtools/plantcare/html), and Softberry NSITE-PL (http://www.softberry.com) to identify putative regulatory motifs. The in silico analysis of the SHOMT2 promoter predicted the presence of several potential cis-acting DNA elements involved in the regulation of gene expression in vascular tissues (Table 2). Motifs previously associated with vascular tissue-specific expression, such as the ASL-box (CTTTA repeat) (Planta 226:429-442, 2007; Plant Journal 12:1179-1188, 1997), Box P (AACCAAAC) (Plant Journal 4:125-135, 1993; Plant Molecular Biology 27:6651-6667, 1995; Plant Science 155:85-100, 2000), BS1 (AGCGGG) (Plant Journal 23:663-676, 2000), NTBBF1 (ACTTTA) (Plant Cell 11:323-334, 1999; Planta 216:824-833, 2003) and AC (ACI (CCTACC), ACII (CCAACC), and ACIII (CCTACC) (Plant Molecular Biology 53:597-608, 2003; Plant Molecular Biology 62:809-823, 2006; Biochemical and Biophysical Research Communications 394:848-853, 2010) were identified in the SHOMT2 promoter (Table 2). The fact that the SHOMT2 promoter is rich with regulatory motifs specific to vascular lignifying cells suggests a functional role for the SHOMT gene in lignification. The SHOMT2 promoter was also found to contain cis-elements conferring responsiveness to the defense-related and stress-responsive hormones, salicylic acid (SA) and the jasmonates, and to abiotic and biotic stresses. These include the ASF1 motif (TGACG) (Plant Journal 11:513-523, 1997; Planta 227:1141-1150, 2008), the T/G box (AACGTG) (Biochimica Biophysica Acta 1679:279-287, 2004; Planta 229:1231-1242, 2009) and the W-box (TTGAC) (Plant Cell Reporter 27:1521-1528, 2008; Planta 227:1141-1150, 2008) (Table 2). The presence of SA- and jasmonate-responsive regulatory elements in the SHOMT2 promoter supports the possible involvement of the SHOMT2 gene in the SA- and jasmonate-induced self-defense responses.

TABLE 2
Putative regulatory motifs enriched in the SHOMT2 promoter

| | | |
|---|---|---|
| | | Occurrence and |
| Name and sequence of motif | Function | position of motif* |

Tissue Specific Motifs

| | | |
|---|---|---|
| BS1 element: AGCGGG | Vascular, stem | 1 (−4686) |
| AC element: CCWWCC | Phloem/xylem; | |
| ACI element: AGCCTACC | phenylpropanoid/lignin | 1 (−441) |
| ACII element: CACCAACC | biosynthesis; elicitor- | 1 (−2834) |
| ACIII element: ATCCATCC | responsive | 1 (−2241) |
| ASL-box: CTTTA repeat | Phloem, shoot, root, | 8 (−754, −1314, |
| | meristem | −2079, −2766, |
| | | −2896, −3360, |
| | | −4278) |
| Box P: MACCWAMC | Vascular, shoot, leaf; | |
| AACCAAAC | phenylpropanoid/lignin | 1 (−3688) |
| | biosynthesis | |
| NTBBF1: ACTTTA | Vascular | 8 (−755, −1315, |
| | | −2080, −2767, |
| | | −2897, −3361, |
| | | −4279) |

Salicylic acid and/or jasmonate-responsive motifs

| | | |
|---|---|---|
| ASF1MOTIF: TGACG | Responsive to | 6 (−2975, −3230, |
| | jasmonates, SA, | −3278, −3352, |
| | biotic and abiotic | −3759, −3798) |
| | stresses | |
| T/G-box: AACGTG | Responsive to | 2 (−2791, −3925) |
| | jasmonates | |
| W-box: TTGAC | Defense-related, | 5 (−1026, −2976, |
| | responsive to | −3758, −3797, |
| | jasmonates, SA and | −4624) |
| | abiotic stresses | |

Motifs were identified by PLACE signal scan
(http://www.dna.affrc.go.jp/PLACE/signalscan.html), PlantCARE motif sampler (http://bioinformatics.psb.ugent.be/webtools/plantcare/html), and Softberry NSITE-PL
(http://www.softberry.com)
* The motif position is given by the number corresponding to the 5′ nucleotide in the motif from the presumed translational start codon (see SHOMT2 promoter sequence SEQ ID NO: 1)

### Example 4

### SHOMT2 Promoter Characterization in Sugarcane

The SHOMT2 promoter has been functionally characterized in planta. Experiments involving the construction of fusions of the SHOMT2 promoter with the β-glucuronidase (GUS) reporter gene, and transfer of these constructs into two agronomic crops, sugarcane and rice, confirmed the high stem-regulated nature of the SHOMT2 promoter. The expression pattern of the SHOMT2 promoter has been studied in different tissues of sugarcane and rice. Stable transformants of sugarcane and rice showing high stem-regulated expression of the GUS gene driven by the SHOMT2 promoter, in the vasculature and storage parenchyma, are available.

An expression vector was produced by cloning the SHOMT2 promoter into a GUSNOS/pUC19 reporter vector (Plant Molecular Biology 32:579-588, 1996) to generate pSHOMT2GUSNOSpUC19 (FIG. 1) for stable transformation of sugarcane. Specifically, the 4.726 kb promoter fragment (SEQ ID NO: 1) was released from the 6.2 kb SHOMT2 genomic clone (FIG. 3) by XbaI/NcoI digestion and ligated as a transcriptional fusion into the XbaI/NcoI-digested vector, GUSNOS/pUC19, replacing the CaMV 35S promoter.

For sugarcane transformation, embryogenic callus cultures were established from young leaf bases and immature flowers of the commercial sugarcane (Saccharum spp. hybrid, cv. CP72-1210) (Plant Cell Reporter 30:13-25, 2011). Transformation of callus by DNA particle gun bombardment and regeneration of shoots were done as described previously (Crop Science 36:1367-1374, 1996; Plant Cell Reporter 30:13-25, 2011). Seven- to forty-week-old calli were bombarded with the pSHOMT2GUSNOSpUC19 (FIG. 1) DNA (4 μg DNA/480 μg particles) and maintained on MS3 medium for seven days in the dark at 28° C. for recovery. Bombarded calli were later broken into small pieces and incubated in the dark at 28° C. on callus induction medium, MS3 with 2,4-dichlorophenoxyacetic acid (3 mg per L) and geneticin (15 mg per L) selection, for a total of four weeks, with sub-culturing every two weeks. For shoot regeneration, calli were grown on MS supplemented with kinetin (2 mg per L), naphthalene acetic acid (NAA) (5 mg per L) and geneticin (15 mg per L) for six to eight weeks under a light (16 h)/dark (8 h) photoperiod. Green shoots of approximately 2 cm in height were transferred into MS rooting medium containing indole-3-butyric acid (4 mg per L) and geneticin (45 mg per L). Rooted plantlets were transferred to potting soil (Metromix, Scotts, Hope, Ark.) in small pots, maintained in an environmental growth chamber at 30° C. under 15 hours of fluorescent and incandescent light for two weeks, and transferred to the greenhouse in 15 cm-diameter pots at 30° C. under natural sunlight.

GUS gene presence and copy number in the transformed sugarcane plants was verified by Southern blot analysis. Genomic DNA was isolated from liquid nitrogen-ground leaf tissues (3 g fresh weight) collected from young leaves of three- to four-month-old sugarcane plants according to Tai and Tanksley (Plant Molecular Biology Reporter 8:297-303, 1990). Genomic DNA (10 μg per lane) was digested overnight with HindIII, electrophoresed on 0.8% (w/v) agarose gels and transferred to Amersham Hybond-XL nylon membranes (GE Healthcare Bio-Sciences Corp., NJ) in an alkaline solution (0.4 M sodium hydroxide) (Sambrook and Russell, Molecular Cloning: A Laboratory Manual, 2001). Pre-hybridization, hybridization, washing and detection of DNA gel blots were performed as described for the Lamda genomic library hybridization (see EXAMPLE 1). HindIII digested genomic DNA from the transformed sugarcane plants was hybridized with a GUS probe. The Southern analysis identified six independent SHOMT2:GUS transgenic sugarcane lines, with most of the lines displaying a multiple hybridization banding pattern (FIG. 4). This indicates that the GUS gene driven by the SHOMT2 promoter has been inserted in multiple copies into the sugarcane genome.

Histochemical localization of GUS expression in the SHOMT2:GUS transgenic sugarcane lines was determined by incubating tissues (stem, leaf and root) in GUS reaction buffer (2 mM 5-bromo-4-choloro-3-indolyl β-D-glucuronide cyclohexylamine salt dissolved in 1% dimethylformamide, 1 mM potassium ferricyanide, 1 mM potassium ferrocyanide, 1 mM EDTA, 50 mM NaPO₄, pH 7.0) at 37° C. for 12 hours, and reaction was stopped with 50 mM phosphate buffer (Jefferson et al., EMBO Journal 6:3901-3907, 1987). Stained plant tissues were photographed with a zoom stereomicroscope (Olympus SZX7, Olympus, Center Valley, Pa.). Quantitative assays of GUS activity (Jefferson et al., EMBO Journal 6:3901-3907, 1987) were performed on sugarcane tissues (stem, leaf and root) as follows. Tissues were homogenized in GUS extraction buffer (50 mM NaPO₄, pH 7.0, 10 mM EDTA, 0.1× sarkosyl, 0.1% Triton X-100 and 10 mM β-mercaptoethanol) and centrifuged for 15 min to collect protein extract. Extract (25 μL for leaf, and 75 μL for stem and root) was incubated with an equal volume of extraction buffer containing 2 mM 4-methlylumbelliferyl β-D-glucuronide (fluorescent substrate) at 37° C. for 60 min, and the reaction was stopped with 0.2 M Na₂CO₃ (950 mL). Fluorescence was measured using a BioRad fluorometer at 365 nm excitation and 460 nm emission wavelengths. Each assay was performed in triplicate. Protein content of extracts was determined using a BioRad Bradford protein assay kit. Data were expressed as pmoles of 4-methylumbelliferone (MU) per min per μg of extracted protein. In order to reduce the error introduced by potential plant to plant variation, GUS gene expression was measured in three different plants regenerated from each independent callus clone. Stem, leaf and roots explants from four-month-old transgenic sugarcane plants were used for histochemical and quantitative biochemical analyses of the GUS reporter gene.

Quantitative analysis indicated that GUS activity levels of the SHOMT2:GUS sugarcane lines were significantly higher is stems than in leaves and roots (Table 3). Stems exhibited 2.7- to 7.5-fold compared to leaves and 1.5- to 2.3-fold compared to roots (Table 3).

TABLE 3
The SHOMT2 promoter drives GUS expression
in the sugarcane stem

| | GUS activity | | |
|---|---|---|---|
| | (pmoles of 4-methylumbelliferone [MU]/min/μg protein) | | |
| Construct | Stem | Leaf | Root |
| SHOMT2:GUS | 84.5 ± 2.4 | 16.1 ± 0.9 | 53.4 ± 1.0 |
| | (40.8-128.2) | (15.1-17.1) | (27.2-56.6) |

Average GUS activity was measured in stems, leaves and roots of four-month-old sugarcane transgenic for SHOMT2:GUS (six independent lines were tested). GUS activity represents three biological samples and three technical repetitions and is reported with the standard error. The range of each set of experiments is indicated in parentheses

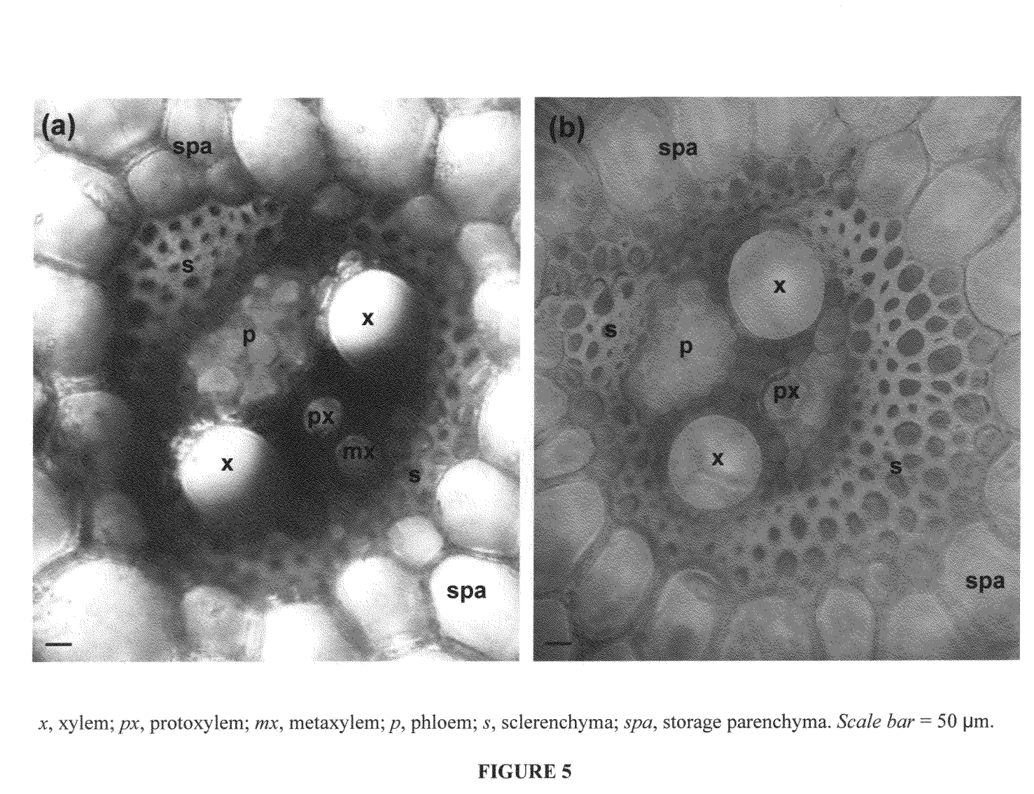

Histochemical analysis showed that GUS expression driven by the SHOMT2 promoter was highly localized in the sugarcane stem vasculature, preferentially in the phloem companion cells, the surrounding bundle sheath cells of the schlerenchymatous tissue, and the cells surrounding the protoxylem and metaxylem (FIG. 5), as well as in the storage parenchyma (FIG. 5). Nontransformed sugarcane tissues showed no GUS expression (FIG. 5).

Histochemical localization of GUS expression directed by the SHOMT2 promoter in situ in sugarcane provides evidence for its activity in the stem, preferentially in the vascular bundle and nodal tissues that participate in the developmentally regulated lignification process. GUS expression directed by the SHOMT2 promoter in the protoxylem suggests that the SHOMT gene is involved in the development of xylem, especially the protoxylem elements that are the first to mature before the surrounding organs have elongated, possibly through activation of secondary cell wall production and lignification. The SHOMT2 promoter is potentially suitable for targeted transgene expression to modify lignin synthesis for improving biomass. In addition, the functional significance of the expression of SHOMT as a structural gene of the phenylpropanoid/lignin pathway in the phloem region lies in its participation in phloem cellular processes. Incorporation of additional phloem-derived cells ensures proper transport of organic nutrients to those cells involved in the reinforcement of the plant axis to counteract the increased weight of the growing plant. Phloem-regulated gene expression can also be beneficial by imposing a decreased metabolic load on the plant. Furthermore, gene expression conferred by the SHOMT2 in the stem storage parenchyma is of great value for metabolic engineering of sugarcane for enhanced carbon metabolism for sugar accumulation or increased fiber content for biofuel feedstock.

### Example 5

### SHOMT2 Promoter Characterization in Rice

An expression vector was produced by cloning the SHOMT2 promoter into the plant binary vector pCAMBIA1301 (CAMBIA, Brisbane, Australia) to generate pSHOMT2 pCAMBIA1301 (FIG. 2) for stable transformation of rice. Specifically, the 4.726 kb promoter fragment (SEQ ID NO: 1) was released from the 6.2 kb SHOMT2 genomic clone (FIG. 3) by XbaI/NcoI digestion and ligated as a transcriptional fusion into the XbaI/NcoI-digested vector, pCAMBIA1310, replacing the CaMV 35S promoter.

For rice transformation, fresh cells of Agrobacterium tumefaciens strain EHA105 (Hood et al., Transgenic Research 2:208-218, 1993) harboring the pSHOMT2 pCAMBIA1301 (FIG. 2) were grown at 28° C. for 30 hours in 1% (w/v) yeast extract, 1% (w/v) peptone and 0.5% (w/v) NaCl medium supplemented with kanamycin (100 μg per mL) and rifampicin (10 μg per mL) (seven individual fresh colonies in 2 mL aliquots). For each construct, cells were pooled, harvested by centrifugation at 735×g, resuspended in 10 mL of pre-induction medium, pH 5.6 (55.5 mM glucose, 75 mM MES, 1×AB salts [20× is 0.37 M NH₄Cl, 50 mM MgSO₄.7H₂O, 40.24 mM KCl, 1.8 mM CaCl₂.2H₂O, 0.18 mM FeSO₄.7H₂O], and 2 mM sodium phosphate pH 5.6) with 100 μM acetosyringone, and grown for an additional 24 hours with shaking. Bacterial suspension at O.D. 600 of 1.5-1.9 was used for rice transformation. Transformation experiments were carried out using embryo-derived calli of rice Taipe±309 variety according to Aldemita and Hodges (Planta 199:612-617, 1996) with certain modifications. Callusing, co-cultivation, regeneration and rooting media compositions were as described (Planta 199:612-617, 1996). Sterilized dehusked seeds were grown on N6 medium with 2 ppm of 2,4-dichlorophenoxyacetic acid for production of embryogenic callus. Six- to eight-week-old mature calli were freshly pre-cultured on N6 medium for 5 days prior to transformation. Co-cultivation of calli with bacterial suspension (10 μL of suspension for each callus) was performed for 3 days in darkness at room temperature on N6 medium supplemented with 55.5 mM glucose and 200 μM acetosyringone. Calli were placed on filter paper overlaid on resting medium (hygromycin-free N6 medium with carbenicillin [250 mg per L] and cefotaxime [100 mg per L]) for one week in darkness at room temperature, before being subjected to selection on N6 medium with hygromycin (50 mg per mL), carbenicillin (250 mg per L) and cefotaxime (100 mg per L) for two rounds of three weeks each. Calli were cultured on fresh selection medium for an additional two weeks and later transferred to regeneration medium (hygromycin-free MS medium with tryptophan [50 mg per L], NAA [0.1 mg per L] and kinetin [2.5 mg per L]) and placed in an environmental growth chamber at room temperature under continuous illumination (about 2000 lux). Green shoots (about 2 cm high), which normally appear in four to five weeks, were transferred to rooting medium (hormone-free MS medium) with hygromycin (30 mg per mL) for two weeks. Plants surviving the final round of selection with well-developed roots were transferred to soil (Redi-earth mix, Scotts, Hope, Ark.) in one-gallon-pots and grown to maturity in the greenhouse at 30° C. under natural sunlight.

The generated rice plants were analyzed by Southern blot as described for sugarcane (see EXAMPLE 2). Pre-hybridization, hybridization, washing and detection of DNA gel blots were performed as described for the Lamda genomic library hybridization (see EXAMPLE 1). HindIII-digested rice genomic DNA was hybridized with a GUS probe. The analysis identified thirteen independent SHOMT2:GUS transgenic rice lines, with most of the lines displaying a single or double hybridization banding pattern (FIG. 6). This indicates that the GUS gene driven by the SHOMT2 promoter has been inserted mostly as a single copy into the rice genome.

Histochemical localization of GUS expression and quantitative assays of GUS activity (Jefferson et al., EMBO Journal 6:3901-3907, 1987) were performed on culm, leaf and root tissues of four-month-old SHOMT2:GUS transgenic rice lines using the same procedures followed for the analysis of the SHOMT2:GUS transgenic sugarcane lines (see EXAMPLE 2).

Quantitative analysis indicated that GUS activity levels of the SHOMT2:GUS sugarcane lines were significantly higher is culms than in leaves and roots (Table 4). Culms exhibited 42.5- to 41.3-fold compared to leaves and 7.8- to 4.1-fold compared to roots (Table 4).

TABLE 4
The SHOMT2 promoter drives GUS expression in the rice culm

| | GUS activity | | |
|---|---|---|---|
| | (pmoles of 4-methylumbelliferone [MU]/min/μg protein) | | |
| Construct | Culm | Leaf | Root |
| SHOMT2:GUS | 287.7 ± 28.2 | 2.5 ± 0.2 | 33.8 ± 2.9 |
| | (276.1-301.8) | (6.5-7.3) | (35.2-74.0) |

Average GUS activity was measured in culms, leaves and roots of four-month-old rice transgenic for SHOMT2:GUS (thirteen independent lines were tested). GUS activity represents three biological samples and three technical repetitions and is reported with the standard error. The range of each set of experiments is indicated in parentheses

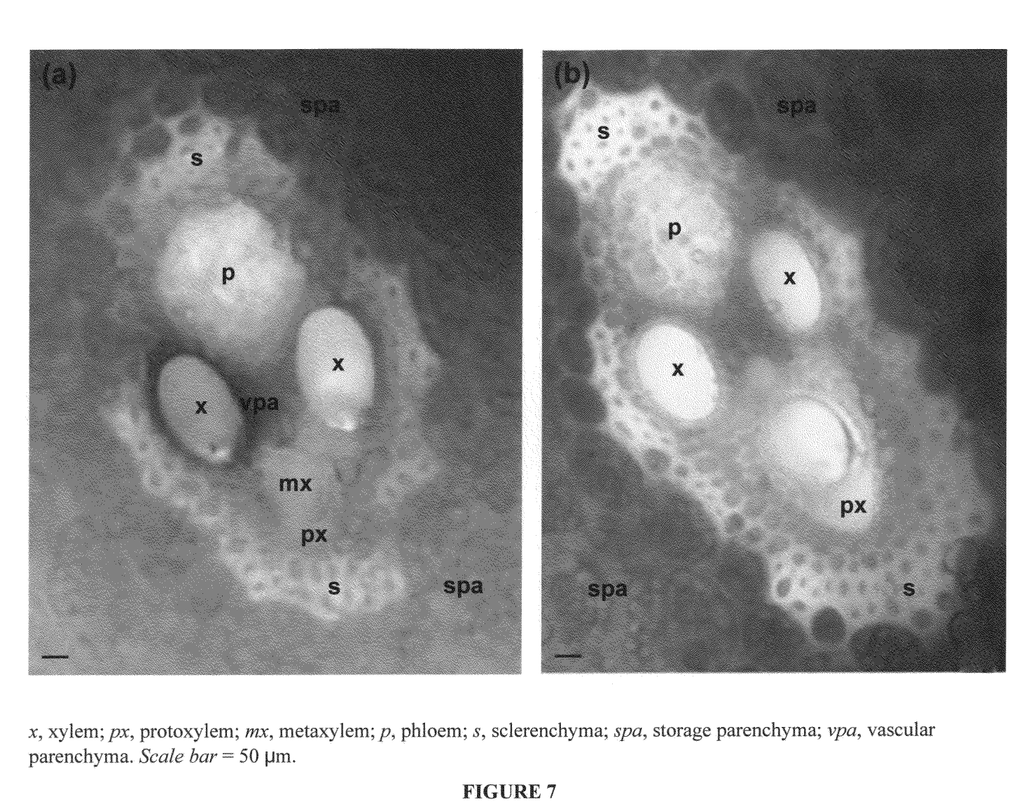

Histochemical analysis showed that GUS expression driven by the SHOMT2 promoter was highly localized in the rice culm vasculature, mainly in the vascular parenchyma surrounding the xylem (FIG. 7), and in the storage parenchyma (FIG. 7). Nontransformed rice tissues showed no GUS expression (FIG. 7).

Even though the pattern of expression in transgenic rice is different than that observed in sugarcane, it closely follows the distribution of cells undergoing lignification. Culm vascular gene expression directed by the SHOMT2 promoter may be exploited to develop plants that are tolerant to important pests and opportunistic fungal pathogens through reinforcement of cell walls of vascular tissues. It may be also useful in developing virus-resistant lines by fusing antiviral constructs to SHOMT2, because many monocot viruses multiply and translocate in the vascular tissue.

### Example 6

### Comparative Expression of SHOMT2 Promoter Relative to Other Promoters

The GUS expression levels driven by the stem-regulated SHOMT2 promoter were compared with those of two previously reported functional stem-regulated promoters, SHOMT (Planta 231:1439-1458, 2010; U.S. Pat. No. 7,323,622; U.S. Pat. No. 7,973,217) and SHDIR16 (Saccharum hybrid dirigent 16) (U.S. Pat. No. 7,253,276; Planta 231:1439-1458, 2010), and of the constitutive maize ubiquitin 1 (UBI1) promoter (Plant Molecular Biology 18:675-689, 1992) in transgenic sugarcane and rice (Table 5).

TABLE 5
Comparative expression levels of GUS driven
by SHOMT2, SHOMT, SHDIR16 and UBI1 promoters
in the sugarcane stem and rice culm

| | GUS activity | | |
|---|---|---|---|
| | (pmoles of 4-methylumbelliferone [MU]/min/μg protein) | | |
| Construct | Stem or culm | Leaf | Root |
| SHOMT2:GUS | | | |
| Sugarcane | 84.5 ± 2.4 | 16.1 ± 0.9 | 53.4 ± 1.0 |
| | (40.8-128.2) | (15.1-17.1) | (27.2-56.6) |
| Rice | 287.7 ± 28.2 | 2.5 ± 0.2 | 33.8 ± 2.9 |
| | (276.1-301.8) | (6.5-7.3) | (35.2-74.0) |
| SHOMT:GUS | | | |
| Sugarcane | 287.0 ± 97.3 | 21.1 ± 11.2 | 29.1 ± 18.6 |
| | (24.9-428.2) | (8.8-43.7) | (11.9-50.6) |
| Rice | 838.2 ± 645.0 | 34.9 ± 34.7 | 31.8 ± 27.6 |
| | (177.0-1466.0) | (4.9-76.8) | (2.1-57.0) |
| SHDIR16:GUS | | | |
| Sugarcane | 1163.2 ± 910.1 | 26.4 ± 18.9 | 42.7 ± 29.9 |
| | (58.0-2073.1) | (12.5-53.0) | (13.0-76.3) |
| Rice | 368.9 ± 306.0 | 33.1 ± 26.4 | 39.0 ± 25.5 |
| | (15.1-551.0) | (2.6-48.3) | (9.6-53.7) |
| UBI1:GUS | | | |
| Sugarcane | 34.2 ± 16.6 | 68.4 ± 17.1 | 58.1 ± 9.0 |
| | (6.0-50.0) | (17.1-93.2) | (37.1-80.1) |
| Rice | 283.6 ± 24.4 | 613.8 ± 45.7 | 728.2 ± 83.0 |
| | (208.0-562.0) | (134.0-1044.0) | (20.4-1642.0) |

Average GUS activity was measured in stems/culms, leaves and roots of four-month-old sugarcane or rice lines (T1) transgenic for SHOMT2:GUS, SHOMT:GUS and SHDIR16:GUS. UB11:GUS lines were included as a positive control. The number of independent SHOMT2:GUS, SHOMT:GUS, SHDIR16 and UB11:GUS transgenic lines tested were six, eight, twelve and four, respectively for sugarcane, and thirteen, eight, thirteen and twelve, respectively for rice. GUS activity represents three biological samples and three technical repetitions and is reported with the standard error. The range of each set of experiments is indicated in parentheses

Quantitative analysis indicated that GUS activity levels of SHOMT2:GUS, SHOMT:GUS and SHDIR16:GUS sugarcane lines were significantly higher in stems than in leaves and roots (Table 5), as compared to UB11:GUS sugarcane lines. GUS activity levels of SHOMT2:GUS sugarcane lines were higher in stems by 2.7- to 7.5-fold compared to leaves and by 1.5- to 2.3-fold compared to roots. Stems from SHOMT:GUS sugarcane lines exhibited 2.8- to 9.8-fold more GUS activity than leaves and 2.1- to 8.5-fold more than roots. Increases in GUS activity of SHDIR16:GUS sugarcane stems were 4.6- to 39.1-fold compared to leaves and 4.5- to 27.1-fold compared to roots. UB11:GUS sugarcane lines displayed higher GUS activity levels in leaves and roots than in stems. Comparative quantitative analysis of GUS expression shows that the SHOMT2 promoter, as the SHOMT and SHDIR16 promoters, confers stem-regulated gene expression in sugarcane, as compared to the UBI1 promoter, which directs gene expression in a constitutive manner. Increases in stem GUS activity levels were lower for SHOMT2:GUS and SHOMT:GUS than for SHDIR16:GUS sugarcane plants.

GUS expression was also confined to culm tissues in transgenic rice harboring SHOMT2:GUS, SHOMT:GUS and SHDIR16:GUS, as compared to UB11:GUS (Table 5). Quantitative analysis revealed higher GUS levels in culms than in leaves and roots of SHOMT2:GUS, SHOMT:GUS and SHDIR16:GUS rice lines (Table 5). Increases in GUS activity of SHOMT2:GUS rice culms were 42.5- to 41.3-fold compared to leaves and 7.8- to 4.1-fold compared to roots. GUS activity in SHOMT:GUS rice culms was 19.1- to 36.1-fold higher compared to leaves and 25.7- to 84.3-fold higher compared to roots. Culms from SHDIR16:GUS rice lines exhibited 5.8- to 11.4-fold more GUS activity than leaves and 1.6- to 10.3-fold more than roots. UB11:GUS rice plants showed higher GUS activity levels in leaves and roots than in culms. Comparative quantitative analysis of GUS expression shows that the SHOMT2 promoter, as the SHOMT and SHDIR16 promoters, confers stem-regulated gene expression in rice, as compared to the UBI1 promoter, which directs gene expression in a constitutive manner. Increases in culm GUS activity levels were higher for SHOMT2:GUS and SHOMT:GUS than for SHDIR16:GUS rice plants.

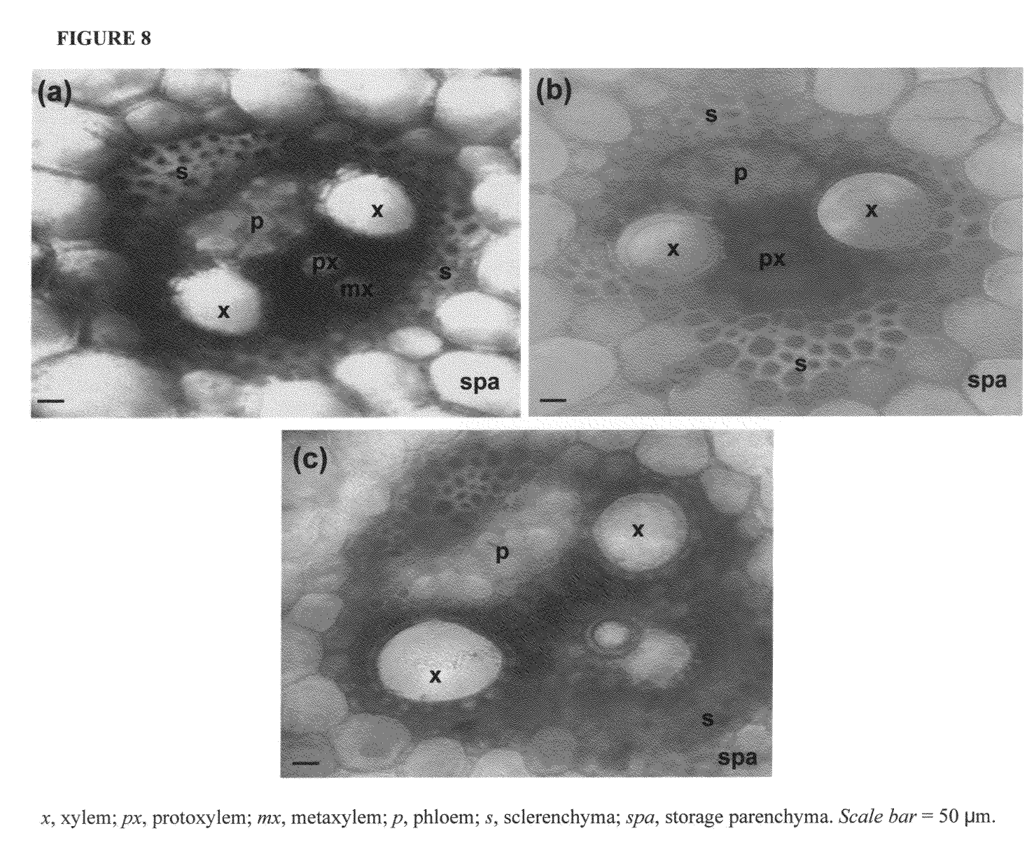

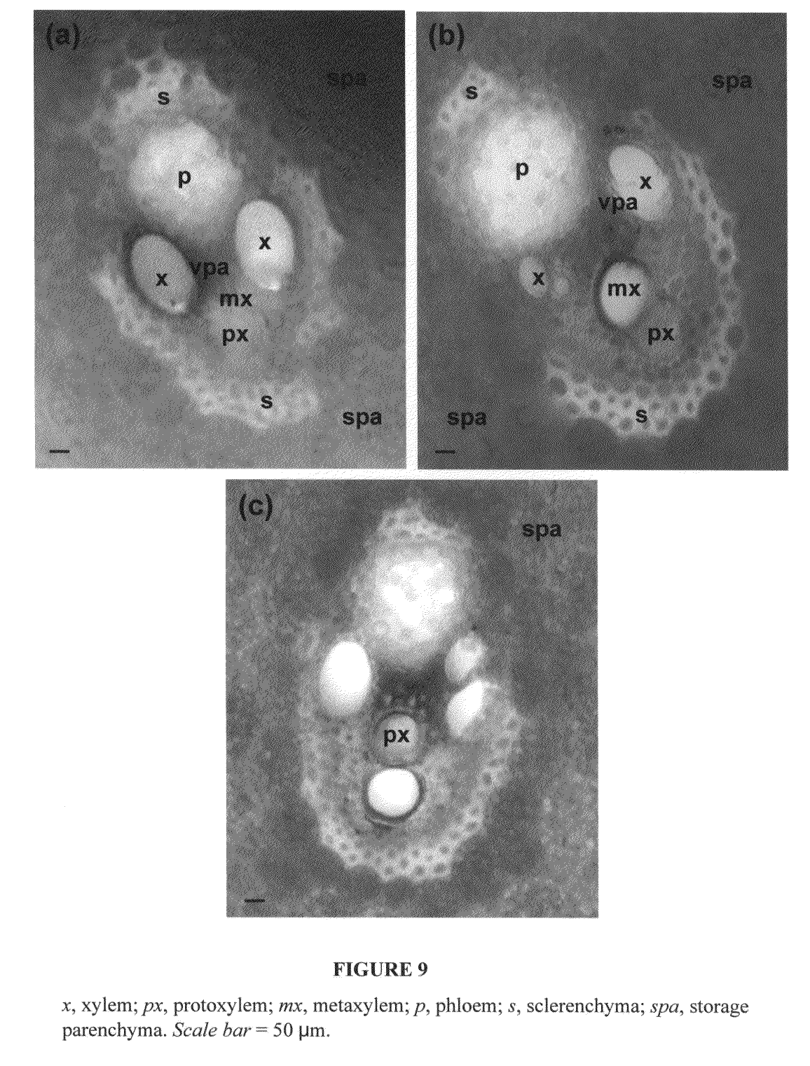

Histochemical analysis of GUS expression driven by SHOMT2, SHOMT and SHDIR16 in sugarcane stem and rice culm revealed that the three promoters conferred GUS expression in the vascular tissues and the storage parenchyma. In sugarcane, GUS expression was associated with the bundle sheath cells of the sclerenchymatous tissue and cells surrounding the protoxylem and xylem for SHOMT2:GUS, SHOMT:GUS and SHDIR16:GUS plants (FIG. 8). Phloem companion cells were also stained for GUS, and staining was more intense in SHOMT2:GUS and SHOMT:GUS than in SHDIR16:GUS sugarcane lines (FIG. 8). Additionally, the SHOMT2 and SHOMT promoters directed GUS expression in the sugarcane stem storage parenchyma, which was more pronounced in the SHOMT2:GUS sugarcane lines FIG. 8A). In rice, the SHOMT2, SHOMT and SHDIR16 promoters conferred a different pattern of GUS expression in the culm vascular system, with significant GUS expression in the vascular parenchyma for SHOMT2:GUS lines and in the protoxylem region for the SHOMT:GUS and SHDIR16:GUS lines (FIG. 9). Interestingly, the SHOMT2 promoter was able to direct GUS expression in the rice culm storage parenchyma (FIG. 9A). Comparative histochemical analysis of GUS expression shows that the SHOMT2 promoter is active in the vascular bundles of the sugarcane stem and rice culm as the SHOMT and SHDIR16 promoters. However, unlike SHOMT and SHDIR16, the SHOMT2 promoter has significant activity in the storage parenchyma of the sugarcane stem and rice culm.

The newly isolated SHOMT2 promoter has specific advantages over the currently available promoters in its enhanced specificity in regulating gene/transgene expression in the stem vasculature and storage parenchyma tissues. At the present time, and compared to other major crops, two functional stem-expressed promoters, SHOMT and SHDIR16 (previously developed by our research group) are only available for use in sugarcane transformation. The development of the SHOMT2 promoter will add to this small repertoire of stem-regulated promoters that are functional (not silenced) in monocots.

### Example 7

### Consensus Sequence of O-Methyltransferase

An expression control sequence in some embodiments, may comprise a nucleic acid having a nucleotide sequence that is about 100% identical to a consensus sequence of SHOMT1 (SEQ ID NO: 4) and nucleotides 1-4726 of SHOMT2 (SEQ ID NO: 1). A consensus sequence was identified using ClustalW (v.1.4) multiple sequence alignment. Settings were selected according to Table 6. Results are shown in FIG. 10.

TABLE 6
Multiple sequence alignment settings
ClustalW (v1.4) multiple sequence alignment
Mac Vector (Mac Vector, Inc., Gary, NC)

| 2 Sequences Aligned | |
|---|---|
| Alignment Score: | 20232 |
| Gaps Inserted: | 6 |
| Conserved Identities: | 2879 |
| Pairwise Alignment Mode: | Slow |
| Pairwise Alignment Parameters: | |
| Open Gap Penalty: | 10.0 |
| Extend Gap Penalty: | 5.0 |
| Multiple Alignment Parameters: | |
| Open Gap Penalty: | 10.0 |
| Extend Gap Penalty: | 5.0 |
| Delay Divergent: | 40% |
| Transitions: | Weighted |
| Sequences: | |
| SHOMT2 vs. SHOMT1 | |
| Aligned Length: | 2956 |
| Gaps: | 6 |
| Identities: | 2879 (97%) |

What is claimed is:

1. An isolated nucleic acid comprising an expression control sequence having the sequence of nucleotides 1-4726 of SEQ ID NO: 1, wherein the expression control sequence has stem-specific and/or defense-inducible promoter activity in at least one monocot.

2. An isolated nucleic acid according to claim 1, wherein the expression control sequence has promoter activity in at least two monocots.

3. An isolated nucleic acid comprising (a) an expression control sequence having the sequence of nucleotides 1-4726 of SEQ ID NO: 1, and (b) an exogenous nucleic acid, wherein the expression control sequence has stem-specific and/or defense-inducible promoter activity in at least one monocot.

4. An isolated nucleic acid according to claim 3, wherein the exogenous nucleic acid comprises a transgene.

5. An isolated nucleic acid according to claim 3, wherein the exogenous nucleic acid alters carbon metabolism in the plant cell when expressed or transcribed.

6. An isolated nucleic acid according to claim 3, wherein the exogenous nucleic acid encodes an insecticide effective against at least one stem-boring insect.

7. An expression vector comprising, in a 5′ to 3′ direction: a sugarcane o-methyltransferase 2 (SHOMT2) promoter having a nucleotide sequence of nucleotides 1-4726 of SEQ ID NO: 1; an exogenous nucleic acid; and a 3′ termination sequence, wherein the SHOMT2 promoter has stem-specific and/or defense-inducible promoter activity in at least one monocot.

8. An expression vector according to claim 7, wherein the exogenous nucleic acid comprises a transgene.

9. An expression vector according to claim 7, wherein the expression vector is located in a bacterial cell.

10. An expression vector according to claim 7, wherein the expression vector is located in a plant cell.

11. A bacterial cell comprising an expression vector having:
a SHOMT2 promoter having a nucleotide sequence of nucleotides 1-4726 of SEQ ID NO: 1;
an exogenous nucleic acid; and
a 3′ termination sequence, wherein the SHOMT2 promoter has stem-specific and/or defense-inducible promoter activity in at least one monocot.

12. A plant cell comprising an expression vector having:
a promoter having a nucleotide sequence of nucleotides 1-4726 of SEQ ID NO: 1;
an exogenous nucleic acid operably linked to the promoter; and
a 3′ termination sequence, wherein the promoter has stem-specific and/or defense-inducible promoter activity in at least one monocot.

13. A plant cell according to claim 12, wherein the exogenous nucleic acid comprises a transgene.

14. A plant cell according to claim 12, wherein the exogenous nucleic acid alters carbon metabolism in the plant cell when expressed or transcribed.

15. A plant cell according to claim 12, wherein the exogenous nucleic acid encodes an insecticide effective against at least one stem-boring insect.

16. A plant cell according to claim 12, wherein the plant cell is located in a plant.

17. A plant cell according to claim 16, wherein the plant is a monocot.

18. A plant cell according to claim 17, wherein the plant is selected from the group consisting of sugarcane, miscanthus, a miscanthus×sugarcane hybrid, switch grass, oat, wheat, barley, maize, rice, banana, yucca, onion, asparagus, sorghum and hybrids thereof.

19. A plant comprising an expression vector having:
a promoter having a nucleotide sequence of nucleotides 1-4726 of SEQ ID NO: 1;
an exogenous nucleic acid operably linked to the promoter; and
a 3′ termination sequence, wherein the promoter has stem-specific and/or defense-inducible promoter activity in at least one monocot.

20. A method for stem-specifically and/or defense-inducibly expressing an exogenous nucleic acid in a monocot, the method comprising:
contacting an expression cassette or expression vector with the cytosol of a cell of the monocot,
wherein the expression cassette or expression vector comprises (i) the exogenous nucleic acid, (ii) a SHOMT2 promoter comprising the sequence of nucleotides 1-4726 of SEQ ID NO: 1 and operable to drive expression of the exogenous nucleic acid in the monocot, and (iii) a 3′ termination sequence operably linked to the exogenous nucleic acid, and
wherein the promoter has stem-specific and/or defense-inducible promoter activity in the monocot.

21. A method according to claim 20, wherein the contacting further comprises biolistically bombarding the cell with a particle comprising the expression cassette or expression vector.

22. A method according to claim 21, wherein the contacting further comprises co-cultivating the cell with an Agrobacterium cell comprising the expression cassette or expression vector.

23. A method according to claim 20, wherein the plant is selected from the group consisting of sugarcane, miscanthus, a miscanthus×sugarcane hybrid, switch grass, oat, wheat, barley, maize, rice, banana, yucca, onion, asparagus, sorghum and hybrids thereof.

24. An isolated nucleic acid comprising an expression control sequence having the sequence of SEQ ID NO: 5, wherein the expression control sequence has stem-specific and/or defense-inducible promoter activity in at least one monocot.

25. An isolated nucleic acid according to claim 24, wherein the expression control sequence has promoter activity in at least two monocots.

26. An isolated nucleic acid comprising (a) an expression control sequence having the sequence of SEQ ID NO: 5, and (b) an exogenous nucleic acid, wherein the expression control sequence has stem-specific and/or defense-inducible promoter activity in at least one monocot.

27. An isolated nucleic acid according to claim 26, wherein the exogenous nucleic acid comprises a transgene.
